(19) United States
(12) Patent Application Publication
(10) Pub. No.: US 2026/0199319 A1
(43) Pub. Date: Jul. 16, 2026

(54) FORCED INDUCTION OF CORNIFICATION TO ERADICATE CANCER AND PREVENT RECURRENCE

(71) Applicant: Fred Hutchinson Cancer Center, Seattle, WA (US)

(72) Inventors: Kevin Jon Cheung, Seattle, WA (US); Andrea E. Doak, Seattle, WA (US); Saki Banjo, Seattle, WA (US); Justin Hui, Seattle, WA (US)

(73) Assignee: Fred Hutchinson Cancer Center, Seattle, WA (US)

(21) Appl. No.: 19/449,186

(22) Filed: Jan. 14, 2026

Related U.S. Application Data
(60) Provisional application No. 63/745,175, filed on Jan. 14, 2025.

(51) Int. Cl. A61K 31/4704; A61K 45/06; A61P 35/00; C12Q 1/6886

(52) U.S. Cl. CPC A61K 31/4704; A61K 45/06; A61P 35/00; C12Q 1/6886; C12Q 2600/106; C12Q 2600/158

(57) ABSTRACT

The present disclosure provides methods for inducing directed and irreversible differentiation of basal-like tumor cells expressing one or more epidermal genes towards a terminal epidermal phenotype. The disclosed methods comprise contacting the basal-like tumor cells with an effective amount of at least one agent that modulates retinoic acid signaling to induce irreversible differentiation of the basal-like tumor cells towards a terminal epidermal phenotype. In some embodiments, the agent modulates retinoic acid signaling by antagonizing a retinoic acid receptor, a retinoic acid signaling component, or a combination thereof. Also provided herein is a method of treating a tumor comprising administering to the subject a therapeutically effective amount of a composition comprising at least one agent capable of inducing directed and irreversible differentiation of the tumor towards a terminal epidermal phenotype. In some embodiments, the tumor comprises basal-like tumor cells expressing one or more epidermal genes.

### CROSS-REFERENCES TO RELATED APPLICATIONS

This application claims the benefit of U.S. Provisional Application No. 63/745,175, filed Jan. 14, 2025, the disclosure of which is incorporated herein by reference in its entirety.

### STATEMENT OF GOVERNMENT LICENSE RIGHTS

This invention was made with Government support under CA234488 awarded by The National Institutes of Health. The Government has certain rights in the invention.

### BACKGROUND

Cancer remains a leading cause of morbidity and mortality worldwide despite substantial advances in diagnostics and therapeutics. Many solid tumors are heterogeneous and comprise multiple cellular subpopulations that differ in morphology, gene expression, differentiation status, and therapeutic responsiveness. This intratumoral heterogeneity contributes significantly to disease progression, therapeutic resistance, and tumor recurrence.

Among these heterogeneous cellular subpopulations are so-called basal-like tumor cells, which have been identified in a variety of malignancies, including but not limited to breast, prostate, lung, and bladder cancers. Basal-like tumor cells are generally characterized by gene expression profiles resembling those of basal epithelial cells or basal epidermal keratinocytes, often including elevated expression of basal cytokeratins, stemness-associated markers, and pathways involved in epithelial-to-mesenchymal transition (EMT). In certain cancer types, tumors enriched in a subpopulation of such basal-like tumor cells are associated with an aggressive phenotype characterized by enhanced proliferation of tumor cells, increased invasiveness, early metastasis, and poor clinical prognosis.

Cancers harboring basal-like tumor cells present particular challenges for existing treatment modalities. For example, basal-like breast tumors are frequently negative for estrogen receptor (ER), progesterone receptor (PR), and HER2/Neu expression, thereby limiting the utility of endocrine and HER2-targeted therapies. Similarly, basal-like subpopulations in other tumor types may lack or downregulate molecular targets that are effectively exploited by current precision medicines. Basal-like tumor cells may also contribute to intrinsic or acquired resistance to standard therapies such as cytotoxic chemotherapy, radiation therapy, hormone-based treatments, and targeted agents.

Current therapeutic strategies generally do not specifically identify, target, or modulate basal-like tumor cells as a distinct cellular population. As a result, patients whose tumors harbor basal-like tumor cells may experience suboptimal responses to therapy and poorer clinical outcomes.

Accordingly, there is an ongoing and unmet need for improved methods and compositions for the detection, characterization, and treatment of cancers that include basal-like tumor cells. In particular, there is a need for therapeutic approaches that can selectively target or modulate basal-like tumor cells, overcome their resistance mechanisms, and improve patient response rates, and overall survival. The present disclosure addresses these needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure addresses the unmet need in the art by recognizing and exploiting the reliance of basal-like tumor cells, specifically basal-like breast carcinoma cells, on retinoic acid signaling pathways. By targeting retinoic-acid signaling, the disclosure provides methods for inhibiting tumor survival, invasion and metastases to specifically target and modulate basal-like tumor cells for treatment of cancers comprising basal-like tumor cells.

In an aspect, the present disclosure provides a method of inducing direct and irreversible epidermal differentiation in a basal-like tumor cell, the method comprising contacting the basal-like tumor cell with an effective amount of at least one agent that modulates retinoic acid signaling. In some embodiments, the basal-like tumor cell expresses one or more epidermal genes. In some embodiments, the method induces directed differentiation of the basal-like tumor cell towards a terminal epidermal phenotype.

In some embodiments, the one or more epidermal genes comprise genes expressed in basal epidermal keratinocytes. In certain embodiments, the one or more epidermal genes are associated with cornified envelope formation, epidermal differentiation, and epidermal morphogenesis. In some embodiments, the one or more epidermal genes comprise cytokeratin-14 (K14), K17, Stratifin (SFN), PERP, DSG3, and LAMA3, Krt6a, and Tp63.

In some embodiments, the basal-like tumor cell is in vitro, ex vivo, or in vivo. In some embodiments, the basal-like tumor cell is part of a dominant population or a subpopulation within a tumor. In some embodiments, the basal-like tumor cell is a carcinoma cell. In certain embodiments, the basal-like tumor cell is derived from a squamous cell carcinoma, breast carcinoma, prostate carcinoma, or pancreatic carcinoma. In an embodiment, the basal-like tumor cell is a breast carcinoma cell, and the breast carcinoma cell is a triple negative breast carcinoma cell, lacking estrogen receptor (ER), progesterone receptor (PR), and HER2/Neu expression.

In some embodiments, the at least one agent modulates retinoic acid signaling by antagonizing a retinoic acid receptor, a retinoic acid signaling component, or a combination thereof. In some embodiments, the at least one agent inhibits retinoic acid signaling. In certain embodiments, the at least one agent inhibits retinoic acid signaling by antagonizing a retinoic acid receptor. In certain embodiments, the retinoic acid receptor is RAR-α, RAR-β, and RAR-γ.

In certain embodiments, the at least one agent is selected from a small molecule, biologic, nucleic acid, and an antibody or a derivative thereof. In some embodiments, the nucleic acid is a siRNA, shRNA, antisense-oligonucleotide, or a CRISPR-based gene editing system.

In some embodiments, the at least one agent is a small molecule. In certain embodiments, the small molecule is selected from BMS493, BMS614, LE135, and LY2955303.

In an embodiment, the at least one agent inhibits retinoic acid signaling by antagonizing a retinoic acid receptor. In certain embodiments, the retinoic acid receptor is the RAR-α. In some embodiments, the agent is a small molecule. In an embodiment, the small molecule is BMS614.

In certain embodiments, the terminal epidermal-phenotype is characterized by an increase in expression of one or more epidermal differentiation markers, and optionally a reduced expression of one or more basal-like tumor cell markers relative to a basal-like tumor cell not contacted with the agent. In some embodiments, the terminal epidermal-like phenotype is further characterized by decreased proliferation, decreased migration, and reduced invasive potential of the basal-like tumor cell relative to a basal-like epidermal cell not contacted with the agent.

In a related aspect, the present disclosure provides a method of treating a tumor in a subject. In some embodiments, the tumor comprises basal-like tumor cells as part of a dominant population or a subpopulation within the tumor. In some embodiments, the tumor comprises basal-like tumor cells expressing one or more epidermal genes. In some embodiments, the one or more epidermal genes comprise genes expressed in basal epidermal keratinocytes. In some embodiments, the one or more epidermal genes comprise genes associated with cornified envelope formation, epidermal differentiation, and epidermal morphogenesis. In some embodiments, the one or more epidermal genes comprise cytokeratin-14 (K14), K17, Stratifin (SFN), PERP, DSG3, and LAMA3, Krt6a, and Tp63.

In some embodiments, the method comprises administering to the subject a therapeutically effective amount of an agent capable of inducing a directed and irreversible differentiation of the basal-like tumor cells towards a terminal epidermal phenotype.

In some embodiments, the terminal epidermal phenotype is characterized by an increased expression of one or more epidermal differentiation markers, and optionally a reduced expression of one or more basal-like tumor markers relative to a basal-like tumor cell of an untreated subject. In some embodiments, the terminal epidermal phenotype is further characterized by upregulation of desmosomal adhesion, keratinization, and adhesion plaques relative to a basal-like tumor cell of an untreated subject.

In some embodiments, the method comprises administering to the subject a therapeutically effective amount of a composition comprising at least one agent that modulates retinoic acid signaling. In some embodiments, the at least one agent is selected from a small molecule, biologic, nucleic acid, and an antibody or a derivative thereof. In an embodiment, the nucleic acid is a siRNA, shRNA, antisense-oligonucleotide, or a CRISPR-based gene editing system. In certain embodiments, the at least one agent inhibits retinoic acid signaling. In certain embodiments, the at least one agent inhibits retinoic acid signaling by antagonizing a retinoic acid receptor. In some embodiments, the retinoic acid receptor is RAR-α, RAR-β, and RAR-γ. In some embodiments, the at least one agent is a small molecule, and wherein the small molecule is selected from BMS493, BMS614, LE135, and LY2955303. In an embodiment, the at least one agent inhibits retinoic acid signaling by antagonizing a retinoic acid receptor. In some embodiments, the retinoic acid receptor is the RAR-α, and the agent is BMS614.

In some embodiments, the method is effective in reducing tumor growth, metastases, and inducing tumor regression.

In certain embodiments, the method further comprises administering at least one other therapeutic agent. In certain embodiments, the at least one other therapeutic agent is selected from a chemotherapeutic agent, an immunotherapeutic agent, an endocrine therapy, or a radiation therapy. In some embodiments, the method is effective in sensitizing the tumor to the at least one other therapeutic agent. In some embodiments, the method is effective in sensitizing the tumor to the chemotherapeutic agent. In an embodiment, the method reduces metastatic spread of the tumor to a secondary tissue.

In some embodiments, the tumor is a squamous cell carcinoma, basal-like breast carcinoma, prostate carcinoma, or pancreatic carcinoma. In an embodiment, tumor is a breast tumor. In some embodiments, the breast tumor is a triple negative breast tumor, lacking expression of estrogen receptor (ER), progesterone receptor (PR), and Her-2/Neu.

In some embodiments, the subject is human.

Also disclosed herein is a pharmaceutical composition comprising at least one agent capable of inducing a directed and irreversible differentiation of basal-like tumor cells towards a terminal epidermal phenotype. In some embodiments, the composition further comprises a pharmaceutically acceptable carrier. In some embodiments, the at least one agent capable of inducing a directed and irreversible differentiation of the basal-like tumor cells towards a terminal epidermal phenotype is an agent that modulates retinoic acid signaling. In certain embodiments, the at least one agent inhibits retinoic acid signaling. In certain embodiments, the at least one agent inhibits retinoic acid signaling by antagonizing a retinoic acid receptor.

In yet another aspect, the disclosure provides a pharmaceutical composition comprising: an agent that modulates retinoic acid signaling and a pharmaceutically acceptable carrier, wherein the composition is formulated to induce terminal epidermal differentiation in basal-like tumor cells. In certain embodiments, the at least one agent inhibits retinoic acid signaling. In certain embodiments, the at least one agent inhibits retinoic acid signaling by antagonizing a retinoic acid receptor.

In an aspect, the disclosure is related to a method of selectively treating a triple negative breast tumor in a subject. In some embodiments, the method comprises obtaining a biological sample of the breast tumor from the subject; detecting basal-like tumor cells expressing a unique transcriptional signature in the biological sample; and administering to the subject a therapeutically effective amount of a composition comprising at least one agent capable of inducing a directed and irreversible differentiation of the basal-like tumor cells towards a terminal epidermal phenotype. In some embodiments, the unique transcriptional signature comprises one or more epidermal genes expressed in basal epidermal keratinocytes. In an embodiment, the one or more epidermal genes comprises genes associated with cornified envelope formation, epidermal differentiation, and epidermal morphogenesis. In some embodiments, the one or more epidermal genes comprise cytokeratin-14 (K14), K17, Stratifin (SFN), PERP, DSG3, LAMA3, Krt6a, and Tp63.

In some embodiments, the at least one agent capable of inducing a directed and irreversible differentiation of the basal-like tumor cells towards the terminal epidermal phenotype is an agent that modulates retinoic acid signaling. In certain embodiments, the at least one agent inhibits retinoic acid signaling. In certain embodiments, the at least one agent inhibits retinoic acid signaling by antagonizing a retinoic acid receptor. In some embodiments, the retinoic acid receptor is RAR-α, RAR-β, and RAR-γ. In some embodiments, the method further comprises administering to the subject at least one other therapeutic agent. In some embodiments, the at least one other therapeutic agent is selected from a chemotherapeutic agent, immunotherapeutic agent, endocrine therapy, and radiation therapy.

In yet another aspect, the present disclosure provides for a method of predicting susceptibility of a tumor in a subject to an agent capable of inducing differentiation of the tumor towards a terminal epidermal phenotype. In some embodiments, the method comprises obtaining a biological sample of the tumor from the subject; and detecting presence of basal-like tumor cells harboring a unique transcriptional signature in the biological sample. In some embodiments, the detection of the basal-like tumor cells harboring the unique transcriptional signature in the biological sample predicts that the tumor is susceptible to the agent.

In some embodiments, the unique transcriptional signature comprises one or more epidermal genes expressed in basal epidermal keratinocytes. In an embodiment, the one or more epidermal genes comprise genes associated with cornified envelope formation, epidermal differentiation, and epidermal morphogenesis. In some embodiments, the one or more epidermal genes comprise cytokeratin-14 (K14), K17, Stratifin (SFN), PERP, DSG3, LAMA3, Krt6a, and Tp63. In some embodiments, the agent capable of inducing an irreversible differentiation of the tumor towards the terminal epidermal phenotype is an agent that modulates retinoic acid signaling. In certain embodiments, the at least one agent inhibits retinoic acid signaling. In certain embodiments, the at least one agent inhibits retinoic acid signaling by antagonizing a retinoic acid receptor. In some embodiments, the retinoic acid receptor is RAR-α, RAR-β, and RAR-γ.

### DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and many of the attendant advantages of this invention will become more readily appreciated as the same become better understood by reference to the following detailed description, when taken in conjunction with the accompanying drawings, wherein:

FIGS. 1A-1L show basal breast tumor cells express epidermal genes in mouse and human breast cancers, in accordance with the present disclosure. FIG. 1A are representative Differential Interference Contrast (DIC) microscopy time-lapse images of MMTV-PyMT mammary tumor organoids invading into 3D collagen I. FIG. 1B shows calculated invasion index of individually tracked organoids over time denoted by the blue thin lines (N=37 organoids, 2 tumors; median index is denoted by the red thick line). FIG. 1C is a representative micrograph of Keratin 14 and DAPI immunofluorescence. FIGS. 1D and 1E show single-cell RNA sequencing (scRNA-seq) analyses performed on invading PyMT mammary tumor organoids at 0, 16, and 72 hours, n=2 biological replicates, specifically the Uniform Manifold Approximation and Projections (UMAPs) for replicate 1 of cell-cycle regressed scRNA-seq data labeled by time (FIG. 1D) and by Krt14 transcript expression (FIG. 1E). FIG. 1F shows all detected genes sorted by correlation of expression with Krt14 expression on per single cell basis. The 1% of genes with the highest correlation are colored in red, while the 1% with lowest correlation are colored in blue. Genes of interest as described herein are labeled. FIG. 1G shows the top 5 gene sets enriched for in the top 1% of genes correlating with Krt14, sorted by p-value (calculated with Metascape). FIGS. 1H to 1K are UMAPs representing gene module scores calculated in Seurat using selected gene sets. Pearson R values denote correlation between each gene module score and Krt14. FIG. 1L is a heatmap showing Krt14 and gene set co-expression in mouse and human tumors, based on scRNA-seq reported from two mouse organoid models, three mouse tumor models, and 47 human breast tumors. Krt14 expression is colored by normalized Krt14/KRT14 expression averaged across all tumor epithelial cells in the sample. Gene set values are colored by correlation of gene module score with Krt14 across all epithelial tumor cells.

FIGS. 2A-2H show retinoic acid signaling supports invasion and viability in MMTV-PyMT organoids, in accordance with the present disclosure. FIG. 2A is a schematic depicting the role of retinoic acid receptors in epidermal differentiation and cornification. FIG. 2B are representative DIC microscopy time-lapse images of MMTV-PyMT mammary tumor organoids invading into 3D collagen I treated with either 1 μM all-trans retinoic acid (AtRA) or 5 μM retinoic acid receptor inhibitor (RARi) BMS493. Due to the short half-life of retinoids, drugs were refreshed every 48 hours. FIG. 2C shows invasion index of MMTV-PyMT organoids exposed to increasing concentrations of AtRA (n≥45 organoids per dose, 3 biological replicates; p-values calculated with Dunnett's multiple comparison test after one way ANOVA). FIG. 2D shows invasion index of MMTV-PyMT organoids exposed to increasing concentrations of RARi (n≥40 organoids per dose, 3 biological replicates; p-values calculated with Dunnett's multiple comparison test after one way ANOVA). FIGS. 2E and 2F show relative viability of organoids exposed to various concentrations of AtRA (FIG. 2E) or RARi (FIG. 2F) for 72 hours (determined by CellTiter-Glo® luminescence). All three biological replicates are plotted as points, while curve is drawn as a spline function. FIGS. 2G and 2H show relative caspase activity of organoids exposed to various concentrations of AtRA (FIG. 2G) or RARi (FIG. 2H) for 72 hours (determined by Caspase-Glo® luminescence). All three biological replicates are plotted as points, while curve is drawn as a spline function. For all figures with p-values indicated by asterisks, * p≤0.05, ** p≤0.01, *** p≤0.001, **** p≤0.0001; ns indicates non-significant comparisons; boxplot represents median, 25th percentile and 75^{th} percentile, whiskers extend from min to max.

FIGS. 3A-3H show epidermal gene expression predicting response to retinoic acid receptor signaling perturbations in mouse and human breast tumor organoids. FIG. 3A are representative endpoint DIC microscopy time-lapse images of basal-like C3 (1)/Tag or luminal MMTV-Neu mammary tumor organoids embedded in 3D collagen at treated with either 1 μM AtRA or 5 μM RARi (BMS493). FIG. 3B shows invasion indices of C3 (1)/Tag and MMTV-Neu organoids treated with either 1 μM AtRA or 5 μM RARi (n≥90 organoids per condition; p-values calculated using Dunn's multiple comparison test after Kruskal-Wallis test; boxplot represents median, 25th percentile and 75th percentile, whiskers extend from min to max. FIG. 3C shows relative CellTiter-Glo® luminescence of C3 (1)/Tag and MMTV-Neu organoids 913 treated with a dose range of RARi. Curve was drawn with a spline equation; n=3 passages of organoids on different days. FIG. 3D shows cornification scores calculated for mouse models of breast cancer using bulk tumor RNAseq using Tukey boxplots. FIG. 3E shows cornification scores calculated for several PDX models of breast cancer using bulk tumor RNAseq. FIG. 3F shows representative endpoint DIC microscopy timelapse stills of TNBC216 and TNBC237 tumor organoids embedded in 3D collagen at treated with either 1 μM AtRA or 5 pMRARi. FIG. 3G shows invasion indices of TNBC216 and TNBC237 organoids treated with either 1 μM AtRA or 5 μM RARi (n≥20 organoids per condition, 3 technical replicates; p-values calculated using Dunn's multiple comparison test after Kruskal-Wallis test; boxplot represents median, 25th percentile and 75th percentile, whiskers extend from min to max). FIG. 3H shows relative CellTiter-Glo® luminescence of TNBC216 and TNBC237 organoids treated with a dose range of RARi. Curve was drawn with a nonlinear inhibition equation in prism; n=3 passages of organoids on different days. For any figure of FIGS. 3A-3H with p-values indicated by asterisks, * p≤0.05, ** p≤0.01, *** p≤0.001, **** p≤0.0001; ns indicates non-significant comparisons.

FIGS. 4A-4C show regulation of collective invasion and viability by genetic and pharmacologic regulators of epidermal differentiation. FIGS. 4A and 4B show top gene programs upregulated (FIG. 4A) and downregulated (FIG. 4B) by 1 μM AtRA exposure for 72 hours in MMTV-PyMT organoids embedded in 3D collagen. Bulk RNAseq data gene set enrichment analysis performed with Metascape. FIG. 4C shows specific epidermal marker transcripts from bulk RNAseq of vehicle and AtRA-treated organoids. For each gene transcript, counts were additionally normalized to the mean of controls (n=4 biological replicates; p-values calculated from differential expression analysis with DEseq R package, * p≤0.05, ** p≤0.01, *** p≤0.001, **** p≤0.0001; ns indicates non-significant comparisons; boxplots represents median, 25th percentile and 75th percentile, whiskers extend from min to max).

FIGS. 5A-5I show retinoic acid receptor inhibition suppresses tumor growth and metastasis in-vivo. FIG. 5A is a schematic depicting timeline for orthotopic transplant experiment. FIG. 5B shows fold change in tumor growth over time in mice treated with either vehicle or BMS493 (n=10 mice per treatment; p-values calculated using Mann-Whitney Test). FIG. 5C shows total macro-metastatic area visible on the outside of the lungs of mice by fluorescent detection of tumor cells at endpoint (n=10 mice per treatment; p-values calculated using Mann-Whitney Test). FIGS. 5D and 5E are representative whole and zoomed images of hematoxylin and eosin (H&E) stained sections of tumors from mice treated with either vehicle (FIG. 5D) or BMS493 (FIG. 5E). Sections marked with “N” depict large necrotic regions. FIG. 5F shows percent of total tumor area that was necrotic in mice, quantified by nuclei density in qupath (n=10 mice per treatment; p-values calculated using Mann-Whitney Test). FIG. 5G shows schematic depicting experimental timelines for prevention and treatment tail-vein experimental arms. FIG. 5H shows total macro-metastatic area visible on the outside of the lungs of mice by fluorescent detection of tumor cells at endpoint (n=5 mice per treatment; p-values calculated using Mann-Whitney test). FIG. 5I shows total macro-metastatic area visible on the outside of the lungs of mice by fluorescent detection of tumor cells at endpoint (n=5 mice per treatment; p-values calculated using Mann-Whitney Test). For all figures of FIGS. 5A-5I with p-values indicated by asterisks, * p≤0.05, ** p≤0.01, *** p≤0.001, **** p≤0.0001; ns indicates non-significant comparisons; boxplots represents median, 25^{th} percentile and 75^{th} percentile, whiskers extend from min to max).

FIGS. 6A-6I show epidermal identity of basal breast cancer cells. FIG. 6A is a schematic depicting how invasion index is calculated from each DIC image. The invasion area is the total area minus the maximum convex area filling the inside of the organoid. The invasion index is the invasion area divided by the total area. FIG. 6B is an UMAP of scRNA-seq data obtained from organoids from replicate 2, colored by time, Krt14 expression, and gene module scores calculated in Seurat. FIG. 6C shows top 5 gene sets enriched for in the bottom 1% of genes correlating with Krt14, sorted by p-value (calculated with Metascape). FIG. 6D shows example correlation plots of two gene module scores with Krt14 expression in MMTV-PyMT organoid cells from both tumors: formation of the cornified envelope (CE), and EMT. FIG. 6E shows cell type assignment of individual cells from invading MMTV-PyMT organoids using unsupervised annotation and the Mouse Cell Atlas. FIG. 6F shows UMAPs and gene set enrichment analysis (GSEA) of top 1% of genes correlating with Krt14 in mouse C3 (1) tag organoids embedded in 3D collagen I. Cornified envelope and smooth muscle scores were calculated. FIG. 6G shows UMAPs and GSEA using scRNA-seq data obtained from MMTV-PyMT de-novo tumors. FIG. 6H shows Pearson R of correlation between cornified envelope score and Krt14 stratified by breast cancer receptor subtype (p-value calculated by one sample t-test. **** indicates p≤0.0001). FIG. 6I shows Pearson R of correlation between cornified envelope score and Krt14 stratified by breast cancer PAM50 subtype (p-value calculated by one sample t-test. * indicates p≤0.05).

FIGS. 7A-7K show migration dynamics. FIG. 7A are representative confocal microscopy time-lapse images of mTmG MMTV-PyMT organoids invading in 3D collagen. Organoids were first sparsely transduced with lentivirus to express H2B-GFP (yellow) while mTomato membranes are shown in magenta. FIGS. 7B-7D are migration tracks of individual cells tracked in confocal time-lapses represented in FIG. 7A (p-values calculated with Wilcoxon test). FIG. 7E shows cumulative distance from start point of cells tracked in confocal time-lapse (p-values calculated with Wilcoxon test). FIG. 7F shows speed of cells over time during migration, measured in microns/min (p-values calculated with Wilcoxon test). FIG. 7G shows directionality of cells over time during migration, representing the propensity of cells to continue in the same direction as the frame before (FIG. 7A) (p-values calculated with Wilcoxon test). FIG. 7H is a schematic depicting cell migration tracks with high directionality and low directionality. FIG. 7I shows average directionality of all cells across all timepoints (p-values calculated with Wilcoxon test).

FIG. 7J shows total distance traveled, in microns, of each cell at endpoint (p-values calculated with Wilcoxon test). FIG. 7K shows average speed (microns/minute) of cells across all timepoints (p-values calculated with Wilcoxon test). For all figures of FIGS. 7A-7K with p-values indicated by asterisks, * p≤0.05, ** p≤0.01, *** p≤0.001, **** p≤0.0001; ns indicates non-significant comparisons. For all FIGS. 7A-7K, n=23 organoids, 352 cells. Boxplots are plotted in the style of Tukey.

FIGS. 8A-8H represent retinoic acid signaling pathway. FIG. 8A is a schematic of retinoic acid signaling pathway. Retinyl palmitate (RP) is often converted to retinol in the liver, then is further processed within cells of various tissues. FIG. 8B shows invasion indices for MMTV-PyMT organoids treated with 9-cis retinoic acid (9-cis-RA) or retinyl palmitate both used at a concentration of 1 μM (n=3 biological replicates, ≥90 organoids per dose; p-values calculated using Dunn's multiple comparison test after Kruskal-Wallis test). FIGS. 8C-8E are UMAPs showing transcription of RARα (FIG. 8C), RARβ (FIG. 8D), and RARγ (FIG. 8E) in scRNA-seq of invading MMTV-PyMT organoids. FIGS. 8F-8H show invasion indices for MMTV-PyMT organoids treated with RARα (FIG. 8F), RARβ (FIG. 8G), and RARγ (FIG. 8H) inhibitors across a dose range (n=3 biological replicates, ≥90 organoids per dose; p-values calculated using Dunn's multiple comparison test after Kruskal-Wallis test). For all figures of FIGS. 8A-8H with p-values indicated by asterisks, * p≤0.05, ** p≤0.01, *** p≤0.001, **** p≤0.0001; ns indicates non-significant comparisons; boxplots represents median, 25th percentile and 75th percentile, whiskers extend from min to max.

FIGS. 9A and 9B show viability curves with all trans-retinoic acid (AtRA) in mouse and human organoids. FIG. 9A shows relative CellTiter-Glo® luminescence of C3 (1)/Tag and MMTV-Neu organoids treated with a dose range of AtRA (curve was drawn with a spline equation; n=3 technical replicates). FIG. 9B shows relative CellTiter-Glo® luminescence of TNBC216 and TNBC237 organoids treated with a dose range of AtRA (curve was drawn with a nonlinear inhibition equation in prism; n=3 technical replicates).

FIGS. 10A-10N show transcriptional changes induced by AtRA and RARi. FIG. 10A shows relative abundance of Cyp26a1 transcripts in MMTV-PyMT organoids treated with a dose range of AtRA or RARi (BMS493) measured with qPCR (p-values calculated with Dunnett's multiple comparison test after one-way ANOVA; n=3 biological replicates). FIG. 10B shows relative abundance of retinoic response gene transcripts in MMTV-PyMT organoids treated with 1 μM AtRA or 5 μM RARi measured with bulk RNAseq (p-values calculated with Dunnett's multiple comparison test after two-way ANOVA (n=4 biological replicates). FIG. 10C is a principal component analysis (PCA) plot of RNAseq samples, indicating that AtRA has a larger transcriptional effect than RARi. Mouse number is followed by either “A1” or “B5” to represent AtRA 1 μM and BMS493 (RARi) 5 μM, respectively. FIG. 10D shows relative abundance of EMT marker gene transcripts in MMTV-PyMT organoids treated with 1 μM AtRA or 5 μM RARi measured with bulk RNAseq (p-values calculated with Dunnett's multiple comparison test after two-way ANOVA; n=4 biological replicates). FIG. 10E shows relative abundance of myoepithelial marker gene transcripts in MMTV-PyMT organoids treated with 1 μM AtRA or 5 μM RARi measured with bulk RNAseq (p-values calculated with Dunnett's multiple comparison test after two-way ANOVA; n=4 biological replicates). FIG. 10F shows selected genes with the highest transcriptional induction following AtRA treatment in PyMT organoids (measured with bulk RNAseq; p-values calculated with Dunnett's multiple comparison test after two-way ANOVA; n=4 biological replicates). FIGS. 10G and 10H are UMAPs depicting gene module scores calculated for single cells in scRNA-seq of invading organoids, based on gene sets created using the top 1% of up- and down-regulated transcripts in MMTV-PyMT organoids treated with AtRA. FIGS. 10I and 10J are UMAPs depicting gene module scores calculated for single cells in scRNA-seq of invading organoids, based on gene sets created using the top 1% of up- and down-regulated transcripts in MMTV-PyMT organoids treated with RARi. FIG. 10K shows qPCR confirmation of changes in keratins after treatment with 1 μM AtRA or 5 μM RARi (p-values calculated with Dunnett's multiple comparison test after two-way ANOVA; n=3 biological replicates per condition). FIG. 10L-10N show relative transcript abundance of Cyp26a1 (FIG. 10L), Krt14 (FIG. 10M), and Krt17 (FIG. 10N) over time in MMTV-PyMT organoids embedded in 3D collagen, measured with qPCR (p-values calculated with Dunnett's T3 multiple comparison test after Welch's ANOVA; n=3 biological replicates per condition with two technical replicates). For all figures of FIGS. 10A-10N with p-values indicated by asterisks, * p≤0.05, ** p≤0.01, *** p≤0.001, **** p≤0.0001; ns indicates non-significant comparisons; bars represent mean of replicates (points) with SD.

FIGS. 11A-11F show epidermal gene expression changes induced by AtRA and RARi. FIGS. 11A and 11B show top gene programs upregulated (FIG. 11A) and downregulated (FIG. 11B) by 5 μM RARi exposure for 72 hours in MMTV-PyMT organoids embedded in 3D collagen. Bulk RNAseq data gene set enrichment analysis was performed with Metascape against human gene programs. FIG. 11C shows enrichment scores of specific gene sets in MMTV-PyMT organoids treated with either AtRA or RARi for 72 hours in 3D collagen (normalized enrichment scores determined by ranking transcripts by t-value and using pre-ranked gene set enrichment analysis). FIGS. 11D-11F are representative confocal images of immunofluorescent detection of Keratin 14 (FIG. 11D), Keratin 17 (FIG. 11E), AE13 hair keratin (FIG. 11F) with quantification (all scale bars represent 100 microns; plots indicate percent of positive cells on a per organoid basis from immunofluorescence (IF) stains; n=3 biological replicates, ≥30 organoids per condition, ≥60,000 total cells; p-values calculated using Dunn's multiple comparison test after Kruskal-Wallis test).

FIGS. 12A-12M show in-vivo experimental data of RARi and AtRA. FIG. 12A is a plot showing number of macro-metastases in each mouse (n=10 mice per treatment; p-values calculated with Mann-Whitney test). FIG. 12B shows start and end weight of mice used in orthotopic tumor study (n=10 mice per treatment; p-values calculated with Mann-Whitney test). FIG. 12C are representative H&E images of mammary glands of mice treated with either vehicle or BMS493. No differences were apparent. FIG. 12D are representative images of keratin 14 in tumors, stained with immunohistochemistry (IHC) (scale bar represents 50 microns). FIG. 12E shows quantification of percent of cells scored as K14-positive (K14+) in tumor sections as calculated with QuPath (p-values calculated with Mann-Whitney test; n=10 mice per condition, each with one tumor section stained). FIG. 12F is a violin plot of cellular K14 DAB intensity in tumors treated with either control of RARi (p-values calculated using Wilcoxon test; medians values are displayed; n=10 mice per condition, each with one tumor section stained). FIG. 12G is a schematic depicting timeline for treatment of de-novo MMTV-PyMT tumors in immunocompetent mice. FIG. 12H shows tumor measurements over time in MMTV-PyMT mice treated with either vehicle or BMS493 (n=3 mice per treatment, at least 5 tumors per treatment). FIG. 12I is a plot of growth rate coefficient of de-novo MMTV-PyMT tumors treated with either vehicle or BMS493 (coefficients derived from fitted model of logarithmic tumor growth; p-values calculated with Mann-Whitney test. n≥9 tumors per treatment, ≥3 mice per treatment). FIG. 12J shows percent of total tumor area determined to be necrotic in de-novo MMTV-PyMT tumors after treatment with vehicle or BMS493 (p-values calculated with Mann-Whitney test; n=3 mice per treatment, one section each). FIG. 12K shows fold change in tumor growth over time in mice treated with either vehicle or AtRA (n=10 mice per treatment; p-values calculated using Mann-Whitney Test; duration of treatment was 3 weeks). FIG. 12L shows total macro-metastatic area visible on the outside of the lungs of mice by fluorescent detection of tumor cells at orthotopic experiment endpoint (n=10 mice per treatment; p-values calculated using Mann-Whitney Test). FIG. 12M shows total macro-metastatic area visible on the outside of the lungs of mice by fluorescent detection of tumor cells at end of prevention tail-vein experiment (n=5 mice per treatment; p-values calculated using Mann-Whitney Test). For all figures of FIGS. 12A-12M with p-values indicated by asterisks, * p≤0.05, ** p≤0.01, *** p≤0.001, **** p≤0.0001; ns indicates non-significant comparisons; boxplots represent median, 25th percentile and 75th percentile, whiskers extend from min to max.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. As used herein, the indefinite articles “a” or “an” should be understood to refer to “one or more” of any recited or enumerated component.

As described herein, any concentration range, percentage range, ratio range or integer range is to be understood to include the value of any integer within the recited range and, when appropriate, fractions thereof (such as one tenth and one hundredth of an integer), unless otherwise indicated.

As used herein, the term “basal-like tumor cell” refers to a cancer cell that exhibits molecular, phenotypic, and functional characteristics associated with a basal or progenitor like state. Such characteristics may include, but are not limited to, expression of basal cytokeratins and/or epidermal genes or epidermal-associated genes, stemminess-associated genes, enhanced plasticity, elevated tumor-initiating capacity, enhanced metastatic potential, and/or resistance to conventional cancer therapies. Basal-like tumor cells within a heterogenous tumor population may be characterized by assessing for the expression of one or more molecular and/or assessing one or more functional characteristics associated with basal or progenitor like state. Basal-like tumor cells may be present as a dominant population or as a subpopulation within a heterogenous tumor.

The term “epidermal genes” and/or epidermal-associated genes as used herein refers to genes related to epidermal lineage commitment, epidermal differentiation and morphogenesis, or epidermal cell identity. The term epidermal genes or epidermal-associated genes may also comprise genes expressed in basal epidermal keratinocytes. Epidermal genes may include keratin genes, genes encoding cell-cell adhesion proteins, genes encoding transcription factors or signaling molecules related to epidermal differentiation, morphogenesis, cornified envelope formation, and lineage specification. Exemplary genes include but are not limited to cytokeratin-14 (K14), K17, Stratifin (SFN), PERP, DSG3, LAMA3, Krt6a, and Tp63. Expression of epidermal genes may be determined at the transcript or protein level.

The term “co-expressed” or “expressed” is used interchangeably and refers to genes or proteins that are expressed (made into functional products) at the same time or in the same cell type, suggesting they might work together in a biological pathway.

The term “terminal epidermal phenotype” refers to stable and persistent differentiation state or an irreversible differentiation, that is maintained despite removal of the inducing agent. It also refers to the cell's permanent commitment to a specialized function, losing its ability to become other cell types. Irreversible differentiation may be evidenced molecularly and/or functionally by assessing one or more of: sustained repression of basal-like gene expression, increased expression of one or more epidermal differentiation markers, decreased proliferation, decreased migration, upregulation of desmosomal adhesion, keratinization, and adhesion plaques, and reduced or loss of invasive potential of the basal-like tumor cells.

The term “cornification signature” and/or “unique transcriptional signature” as used herein refers to detectable pattern of expression, regulation, or activity of one or more nucleic acid sequences, or gene products associated with epidermal differentiation, epidermal morphogenesis, cornified envelope formation, and terminal keratinocyte cornification wherein the detected pattern is specific for basal-like tumor cells undergoing or predisposed to undergo, epidermal differentiation, as compared to tumor cells not expressing the one or more genes. The unique transcriptional/cornification signature may comprise increased or decreased expression of one or more genes associated with cornified envelope formation, epidermal differentiation, and epidermal morphogenesis.

The term “agent” refers to any compound, small molecule, biologic, nucleic acid, cell-based therapy, an antibody or a derivative thereof, or combination thereof capable of inducing irreversible differentiation and/or a terminal epidermal phenotype in basal-like tumor cells.

The term “retinoic acid signaling” refers to cellular signaling mediated by retinoids including retinoic acid and its derivatives, through retinoic acid receptors (RARs), retinoid X receptors (RXRs) or downstream signaling pathways and transcriptional mechanisms regulated thereby.

“Administering” refers to the physical introduction of a composition comprising a therapeutic agent to a subject, using any of the various methods and delivery systems known to those skilled in the art. Preferred routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Alternatively, the agents disclosed herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

A “subject” includes any human or nonhuman animal. The term “nonhuman animal” includes, but is not limited to, vertebrates such as nonhuman primates, sheep, dogs, cats, rabbits, ferrets, rodents such as mice, rats and guinea pigs, avian species such as chickens, amphibians, and reptiles. In preferred embodiments, the subject is a mammal such as a nonhuman primate, sheep, dog, cat, rabbit, ferret or rodent. In more preferred embodiments, the subject is a human. The terms, “subject,” “patient” and “individual” are used interchangeably herein.

An “effective amount” as used herein, means an amount which provides a desired effect, or a therapeutic or prophylactic benefit.

A “therapeutically effective amount” or “therapeutically effective dosage” of an agent, such as the agents disclosed herein, is any amount of the agent that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in vitro assays and xenografts.

“Treatment” or “treating,” as used herein, includes any desirable effect on the symptoms or pathology of a disease or condition, e.g., breast cancer, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art. To “treat” a disease as the term is used herein, means to reduce the frequency or severity of at least one sign or symptom of a disease or disorder experienced by a subject.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition that is usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

A “decreased” or “reduced” or “lesser” amount is typically a “statistically significant” amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein.

Reference throughout this specification to “an embodiment” or “one embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Various aspects of the disclosure are described in further detail below.

The present disclosure is based on the discovery of basal-like tumor cells as a distinct set of cells within a heterogenous cell population of established solid tumors and vulnerability of these cells to be leveraged for a targeted or directed therapeutic attack. The present disclosure provides methods for inducing directed irreversible epidermal differentiation in basal-like tumor cells that express epidermal genes or epidermal-associated genes, as well as methods of treating cancers harboring such cells. Without being bound by theory, it is believed that basal-like tumor cells expressing epidermal genes exist in a plastic state that can be therapeutically redirected to a stable or a terminal epidermal phenotype. Inducing irreversible differentiation in these cells leads to decreased proliferation, migration, and invasive potential-leading to decreased tumorigenic potential.

The present disclosure provides methods for inducing directed irreversible epidermal differentiation of basal-like tumor cells expressing one or more epidermal genes through modulation of retinoic acid signaling. In some embodiments, the method comprises contacting the basal-like tumor cell with an effective amount of at least one agent that modulates retinoic acid signaling. The present inventors have discovered that basal-like tumor cells exhibiting/expressing epidermal genes or epidermal-associated genes are particularly responsive to retinoic-acid mediated differentiation resulting in differentiation of the basal-like tumor cell towards a terminal epidermal phenotype that suppresses malignant properties.

Without being bound by theory it is believed that modulating retinoic acid signaling engages endogenous epidermal differentiation programs in basal-like epidermal tumor cells that is different from epithelial-mesenchymal transition (EMT), thereby driving these cells out of plasticity towards a terminal epidermal phenotype.

Unlike conventional cytotoxic approaches that aim to kill tumor cells, the methods disclosed herein modulate cellular identity and fate-thereby rendering the cells less proliferative, less plastic, and less capable of sustaining malignant growth.

In some embodiments, the basal-like tumor cells suitable for treatment express one or more epidermal genes and may exhibit detectable expression or activity of retinoic acid signaling pathway, including retinoic acid receptors (RARs), retinoid x receptors (RXRs), or retinoid responsive transcriptional regulators or other molecules/components involved in retinoic acid mediated signaling and downstream pathways. Expression of epidermal genes and retinoic acid signaling components may be assessed by methods known in the art, including but not limited, to gene expression assessment, immunohistochemistry, or functional assays.

Tumor cells comprise a heterogeneous population of cells. Certain tumor cells despite retaining malignant and progenitor-like properties may express one or more epidermal genes or epidermal-associated genes, including but not limited to keratin family genes associated with cornified envelope formation, epidermal differentiation and epidermal morphogenesis. Such an expression may reflect plasticity that may be potentially selectively leveraged for inducing terminal epidermal phenotype or irreversible differentiation of the tumor cells. Expression may be detected by gene expression profiling, immunohistochemistry, in situ hybridization single cell RNA sequencing or other methods known in the art.

The inventors identified basal-like tumor cells within heterogenous tumor cell populations by single-cell RNA sequencing. Example 1 provides methods used to identify basal-like tumor cells based on expression of one or more basal epithelial or epidermal related genes including cytokeratin-14 (K14), K17, Stratifin (SFN), PERP, DSG3, LAMA3, Krt6a, and Tp63, KRT5, KRT6A. The one or more epidermal genes are associated with cornified envelope formation, epidermal differentiation, and epidermal morphogenesis.

Also provided herein are pharmaceutical compositions comprising at least one agent capable of inducing a directed and irreversible differentiation of basal-like tumor cells towards a terminal epidermal phenotype; and a pharmaceutically acceptable carrier.

Agents useful for the disclosed methods and compositions may include any agent or a combination of agent capable of inducing a directed and irreversible differentiation of basal-like tumor cells of a tumor towards a terminal epidermal phenotype.

Agents may be small molecules, biologics, peptides, nucleic acids, gene-editing constructs, antibodies, or combinations thereof. Exemplary nucleic acid agents may include but are not limited to siRNA, shRNA, antisense oligonucleotide, or a CRISPR-based gene editing system. The disclosure is not limited to any particular agent, provided that the functional outcome of inducing a terminal epidermal phenotype is achieved.

In some embodiments, the disclosure provides a pharmaceutical composition comprising an agent that modulates retinoic acid signaling. In some embodiments, the composition further comprises a pharmaceutically acceptable carrier.

Modulating retinoic acid signaling may comprise blocking synthesis of retinoic acid, blocking binding of retinoic acid to its receptors, and/or modulating downstream effectors of retinoic acid signaling pathways. In some embodiments, the agent capable of directed and irreversible differentiation of basal-like tumor cells of a tumor towards a terminal epidermal phenotype is an agent that inhibits retinoic acid signaling. The agent may inhibit retinoic acid signaling by antagonizing a retinoic acid receptor, a retinoic acid signaling component, or a combination thereof. In some embodiments, the agent inhibits retinoic acid signaling by antagonizing a retinoic acid receptor. In some embodiments, the retinoic acid receptor is RAR-α, RAR-β, and RAR-γ. In some embodiments, the at least one agent is a small molecule. The small molecule may be selected from BMS493, BMS614, LE135, and LY2955303.

A terminal epidermal phenotype induced by the methods of the present disclosure may be characterized by one or more of functional and/or molecular criteria, relative to a basal-like tumor cell not contacted with the agent(s) disclosed herein. Exemplary functional and/or molecular criteria include but are not limited to: increased expression of one or more epidermal differentiation markers; reduced expression of one or more basal-like tumor cell markers; decreased proliferation; reduced migration, and loss of or reduction of invasive potential of the basal-like tumor cell. In some embodiments, the terminal epidermal phenotype is further characterized by upregulation of desmosomal adhesion, keratinization, and adhesion plaques.

Assessment of induction of terminal epidermal phenotype or irreversible differentiation may be assessed by using assays known in the art, including but not limited to, gene expression assays, protein expression analysis, invasion assays, xenograft models, epigenetic profiling. Assessment of functional and/or molecular criteria of terminal phenotype or irreversible phenotype may be assessed relative to a tumor comprising basal-like tumor cells or basal-like tumor cells not contacted with the agents disclosed herein.

The disclosure further provides methods of treating a tumor in a subject by administering one or more agents that induces a directed and irreversible differentiation of the tumor towards a terminal epidermal phenotype or induces irreversible differentiation.

Many solid cancers arise in the setting of arrested differentiation and the degree of differentiation of such tumors correlates with the clinical aggressiveness of disease, forming the basis of current tumor grading schemas. Indeed, the most concerning tumors are referred to as anaplastic, denoting cells that have lost many or all the characteristics of their tissue-type of origin. Cell fate switching is also recognized as an effective mechanism by which tumors evade chemotherapy.

Tumors suitable for treatment according to the disclosed methods may include heterogeneous tumors in which basal-like tumor cells constitute a subpopulation. In some embodiments, the basal-like tumor cells within the heterogenous tumor contribute to aggressiveness, resistance to therapy, increased metastatic potential and recurrence of the tumor. Exemplary tumors include but are not limited to squamous cell carcinoma, breast carcinoma, prostate carcinoma, or pancreatic carcinoma.

The disclosure provides methods of treating a tumor in a subject by administering a therapeutically effective amount of an agent that inhibits retinoic acid signaling. Inhibiting retinoic acid signaling may comprise blocking synthesis of retinoic acid or blocking binding of retinoic acid to its receptors. The agent may inhibit retinoic acid signaling by antagonizing a retinoic acid receptor, a retinoic acid signaling component, or a combination thereof. In some embodiments, the agent inhibits retinoic acid signaling by antagonizing a retinoic acid receptor. In some embodiments, the retinoic acid receptor is RAR-α, RAR-β, and RAR-γ. In some embodiments, the at least one agent is a small molecule. The small molecule may be selected from BMS493, BMS614, LE135, and LY2955303.

In some embodiments, the tumor is a solid tumor comprising a heterogenous population of tumor cells, including basal-like tumor cells. In some embodiments, the agent selectively targets the basal-like tumor cells by forcing differentiation rather than cytotoxicity.

In some embodiments, the tumor is a solid tumor. Non-limiting examples include breast cancer, prostate cancer, bladder cancer, squamous cell carcinoma, and pancreatic carcinoma. In some embodiments, the tumor contains a heterogenous mixture of basal-like tumor cells and more differentiated tumor cells. In some embodiments, the tumor is a breast cancer tumor. In certain embodiments, the tumor is a triple negative breast tumor lacks estrogen receptors (ER), progesterone receptors (PR), and HER2 protein.

Administration of the agent may be systemic or local and may be performed according to dosing regimens determined by clinical considerations.

Treatment may result in one or more therapeutic benefits, including reduction in tumor growth, reduction in recurrence, reduction in metastatic potential, or depletion of basal-like tumor cells.

In some embodiments, the disclosed methods are used in combination with one or more additional anti-cancer therapies, including chemotherapy, radiation therapy, immunotherapy, hormonal therapy, or targeted therapy. Induction of directed and irreversible differentiation of the tumor towards a terminal epidermal phenotype may sensitize tumors to additional therapies by reducing plasticity, proliferation, decreasing the population of basal-like epithelial cells in the tumor, or reducing invasive potential of the tumor.

The one or more therapies may be administered prior to, concurrently with or following the administration of the composition of the disclosure. In some embodiments, the agent capable of inducing the terminal epidermal phenotype is utilized as the primary therapy followed by a secondary therapy. In some embodiments, the secondary therapy is selected from one or more of chemotherapy, radiation therapy, immunotherapy, hormonal therapy, or targeted therapy. In some embodiments, the primary therapy sensitizes the tumor to the secondary therapy.

The disclosure also provides methods of predicting susceptibility of a tumor in a subject to an agent capable of inducing differentiation of the tumor towards a terminal epidermal phenotype. In some embodiments, the method comprises obtaining a biological sample of the tumor from the subject; and detecting presence of basal-like tumor cells harboring a unique transcriptional signature in the biological sample. In some embodiments, the detection of the basal-like tumor cells harboring the unique transcriptional signature predicts that the tumor is susceptible to the agent. In some embodiments, the unique transcriptional signature comprises one or more epidermal genes associated with cornified envelope formation, epidermal differentiation, and epidermal morphogenesis.

In certain embodiments, subjects are selected for treatment based on the presence of basal-like tumor cells expressing one or more epidermal genes or epidermal-associated genes. In some embodiments, subjects are selected for modulation of retinoic acid signaling based on detection of basal-like tumor cells expressing one or more epidermal genes. Detection may be performed using biological samples obtained from the subjects. The methods of obtaining a biological sample may include methods of biopsy including fine needle aspiration, core needle biopsy, vacuum assisted biopsy, incisional biopsy, excisional biopsy, punch biopsy, shave biopsy, or skin biopsy. The biological sample may include tissues including but not limited to skin, heart, lung, kidney, breast, pancreas, liver, muscle, smooth muscle, bladder, gall bladder, colon, intestine, brain, prostate, esophagus, or thyroid. Alternatively, the sample may be obtained from any other source including but not limited to blood, sweat, hair follicle, buccal tissue, tears, or saliva.

A biological sample may be any material containing tissues, cells, nucleic acids, genes, gene fragments, expression products, gene products (e.g., mRNA or proteins), or gene product fragments of a subject to be tested. A biological sample may include but is not limited to, tissue, cells, or biological material from cells or derived from cells of an individual.

The selection or stratification may improve therapeutic outcomes by identifying subjects most likely to benefit from the methods disclosed herein.

### EXAMPLES

The following examples are provided to supplement the above disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

Highly plastic tumors harbor a continuum of cellular states, some of which confer selective advantages during critical phases of local invasion and metastatic spread. A key goal of cancer biology research is to uncover the molecular architecture of cell plasticity and in turn, devise ways to control or eradicate these cells, thereby limiting metastasis and mortality.

Collective cell migration has emerged as a major mechanism for the local spread of cancer cells across solid tumor types and is associated with distant metastasis. During collective cell invasion, leading cancer cells display distinct molecular properties, such as states along epithelial-mesenchymal transition (EMT) axis, or expression of a basal epithelial lineage program. In addition to guiding collective cell migration, breast tumor cells with basal phenotype also support early metastatic seeding and colonization. In treatment contexts, basal-like tumor cells become enriched upon chemotherapy and endocrine therapy and act as a reservoir for drug resistance and tumor regrowth. Among breast cancer subtypes, basal-like tumor cells are particularly enriched in triple-negative breast cancers (TNBC) 30-32, which have poor clinical prognosis and propensity to metastasize. Together, these findings highlight the potential benefit of therapeutically targeting basal tumor cells to better eradicate breast tumors and prevent their successful metastasis. Yet compared with EMT states, the key molecular vulnerabilities of basal tumor cells are not understood.

The present disclosure pertains to the dynamics of cell plasticity giving rise to basal leader cells in breast cancer. Through integrating mouse and human breast tumor organoid models, mouse models of metastasis, and clinical epidemiologic evidence, a skin epidermal-like cell state has been uncovered, particularly enriched in triple-negative breast tumors, rendering these tumor cells exquisitely sensitive to retinoic acid receptor signaling mediated differentiation and keratinization-associated cell death.

Tumor cell plasticity is linked to metastatic aggression and therapy resistance, but could also introduce latent vulnerabilities amenable to therapeutic attack. In the present disclosure, single cell transcriptomics in invasive breast tumor organoids were employed to reveal the molecular cartography of basal epithelial plasticity. This revealed that basal breast tumor cells harbor a transcriptional program resembling skin epidermis. In turn, this molecular map yielded a prediction for directing tumor cellular state transitions. By perturbing epidermal differentiation programs pharmacologically or genetically, potent changes were induced in basal tumor invasion, proliferation, and cell survival. Thus, it is concluded that in basal breast tumors, RAR signaling is a molecular rheostat that promotes a hyper-migratory and proliferative phenotype, and when suppressed, induces terminal epidermal differentiation, hyperkeratinization, and acute cell death.

In addition, this study advances recent conceptual understanding of cell plasticity states and their relation to collective migration. All-trans retinoic acid (AtRA) induces increased directed cell migration and a hypermigratory phenotype, yet this molecular transition appears distinct from classical Epithelial-Mesenchymal Transition (EMT). The importance of intermediate states along the EMT spectrum is relevant for understanding dynamics along the continuum of metastatic stages. The presently described data suggests the existence of cellular states along an epidermal differentiation spectrum that variably contribute to collective invasion, and that potentially links tumor cell migration to programs of epidermal wound repair. In addition, recent studies indicate the benefit of pushing mesenchymal cells past the point of no return-such as via PPARy agonists to drive metastatic cells toward adipocyte differentiation. The data described herein show that antagonizing the RAR signaling pathway induces a similarly favorable effect by irreversibly directing basal breast tumor cells toward an epidermal differentiated fate, thereby limiting their invasive and metastatic potential.

The present disclosure provides evidence that patients with basal-like breast tumors, a molecular phenotype particularly enriched among poor-prognosis triple-negative breast cancers, are candidates for RAR antagonists and other therapies under the umbrella of cornification-inducing therapies or directed irreversible differentiation. In other tumor types, such as acute promyelocytic leukemia and pediatric neuroblastoma, retinoic acid is a potent differentiation-inducing agent. Different therapeutic paradigms ought to be applied when considering distinct tumor context, cell-of-origin, and developmental potential.

### Example 1. Basal Breast Tumor Cells Co-Express Epidermal Genes in Mouse and Human Breast

When breast tumor organoids are embedded in a 3D collagen I matrix, cells at the tumor matrix interface gain migratory features and invade collectively into the surrounding microenvironment. It has been shown previously that leader cells in both human and mouse breast cancers can arise from phenotypic plasticity in which cells acquire a basal epithelial molecular phenotype.

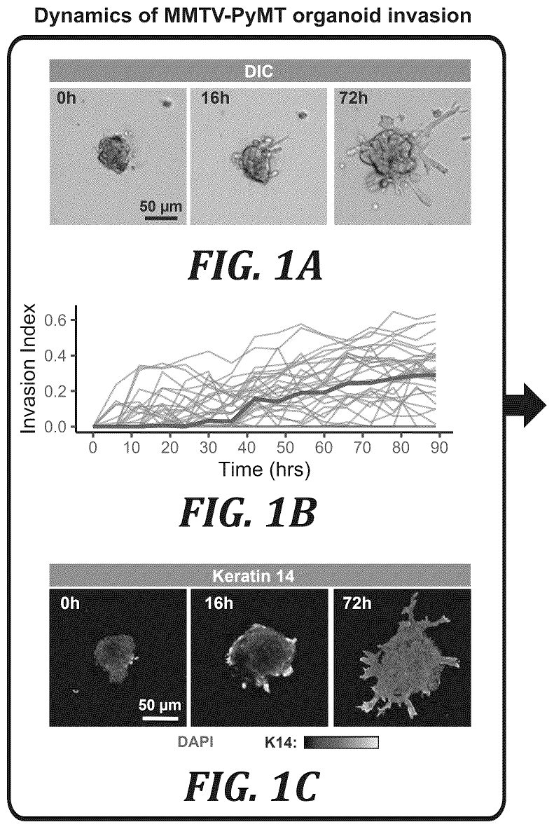

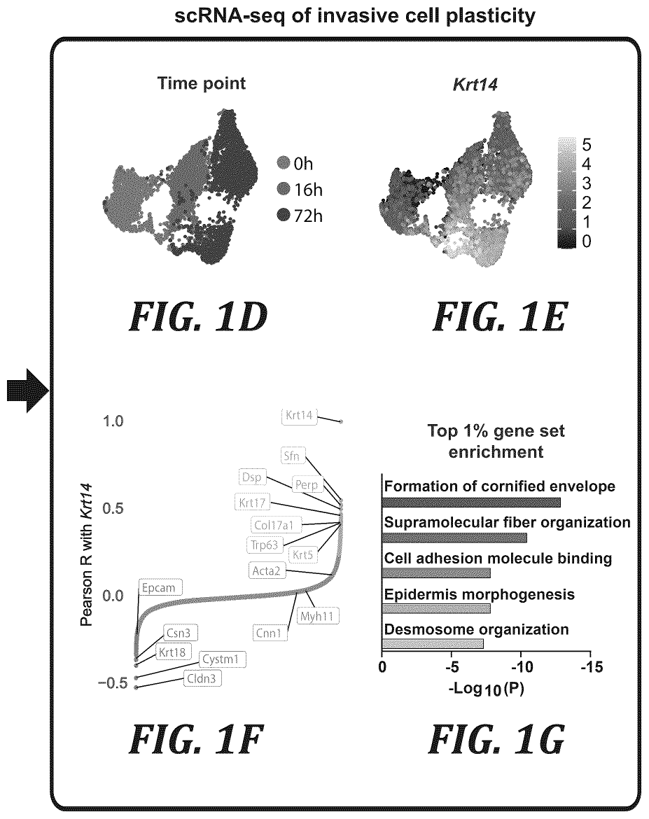

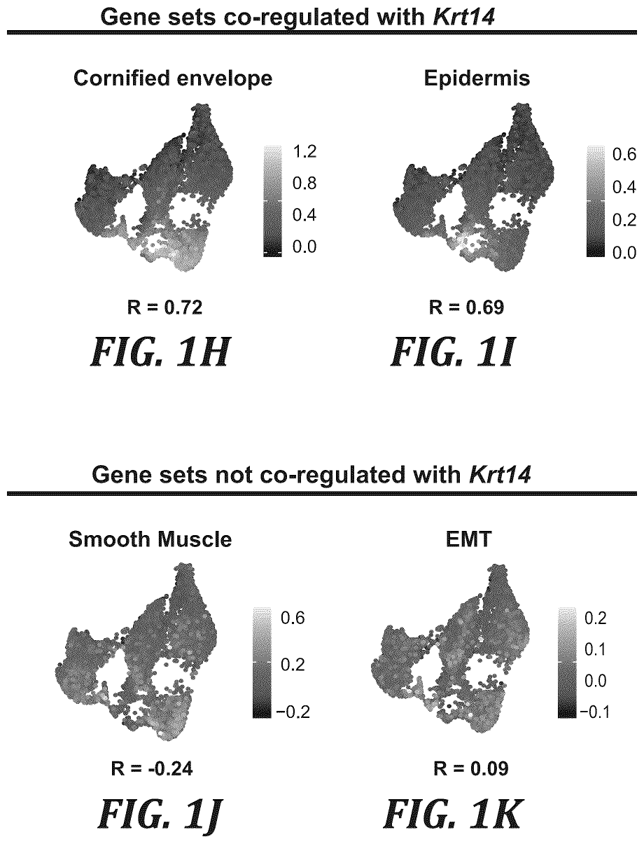

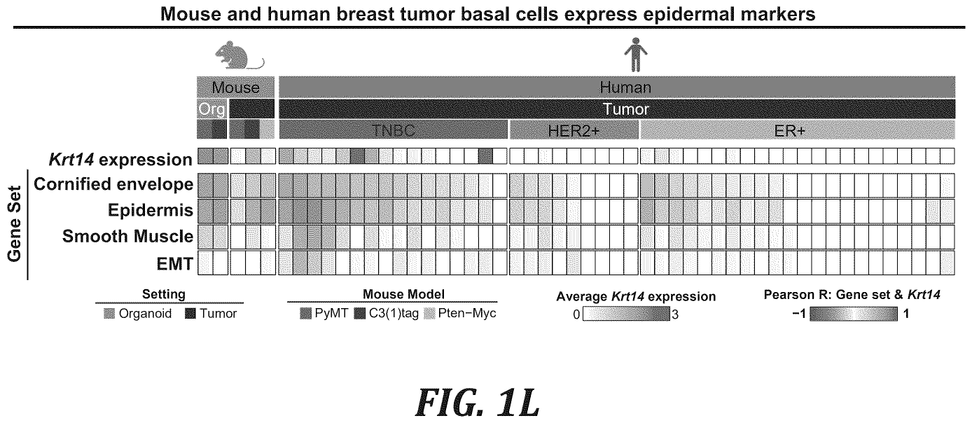

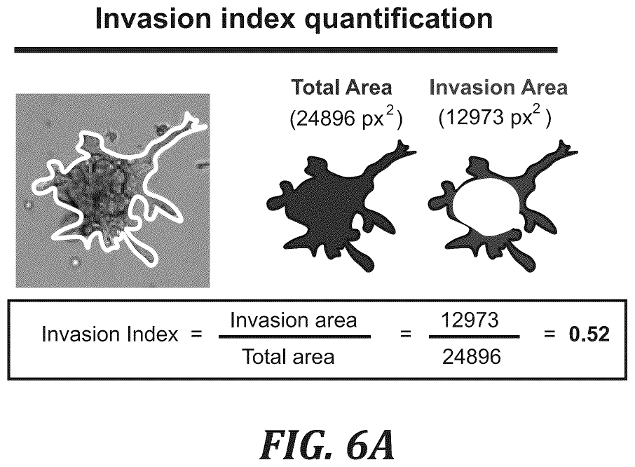

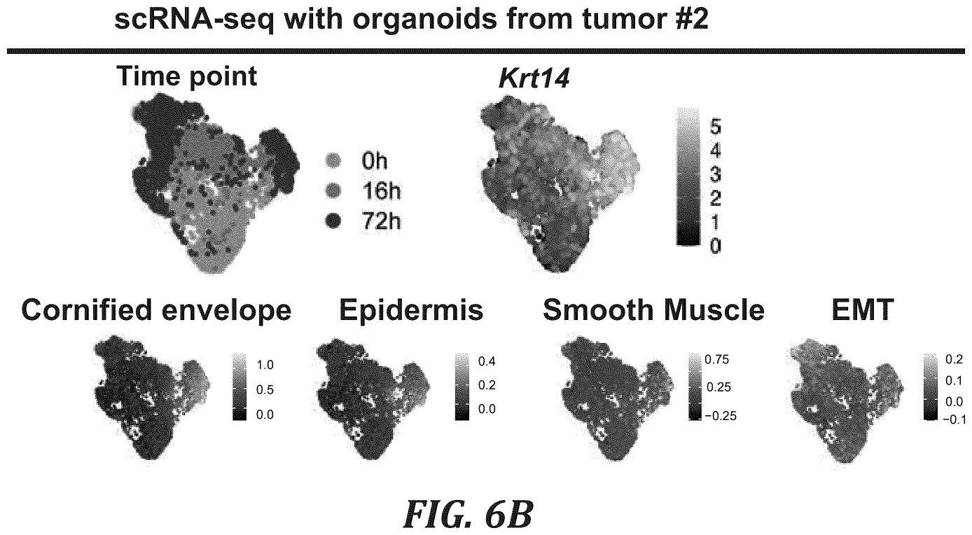

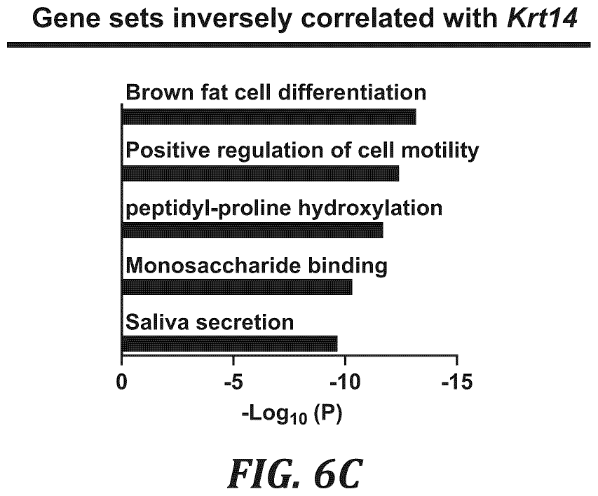

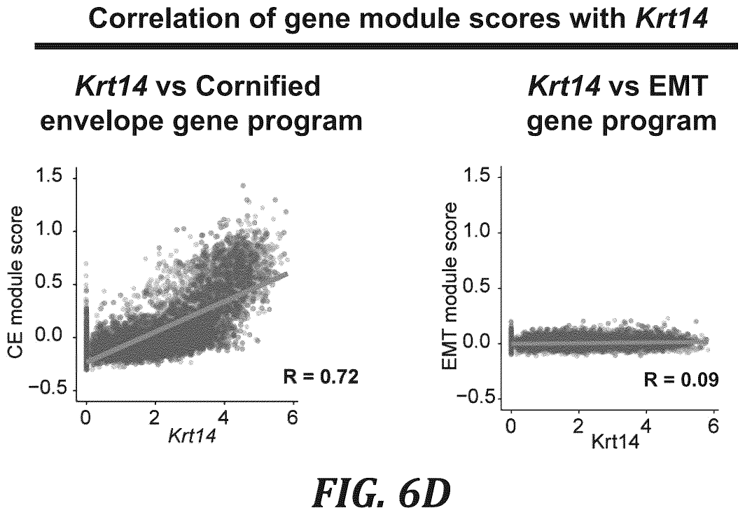

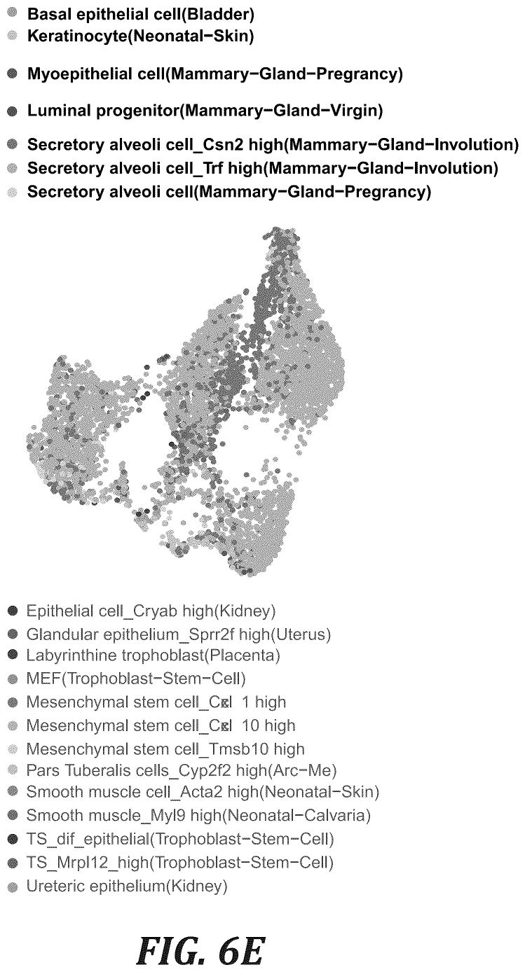

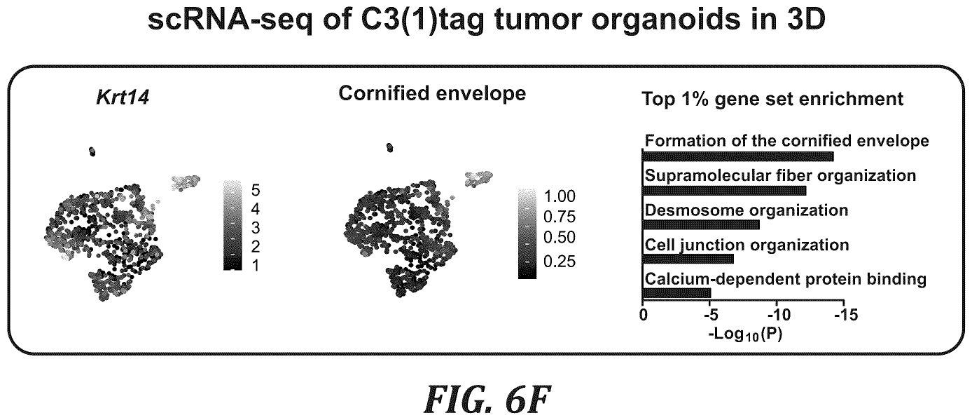

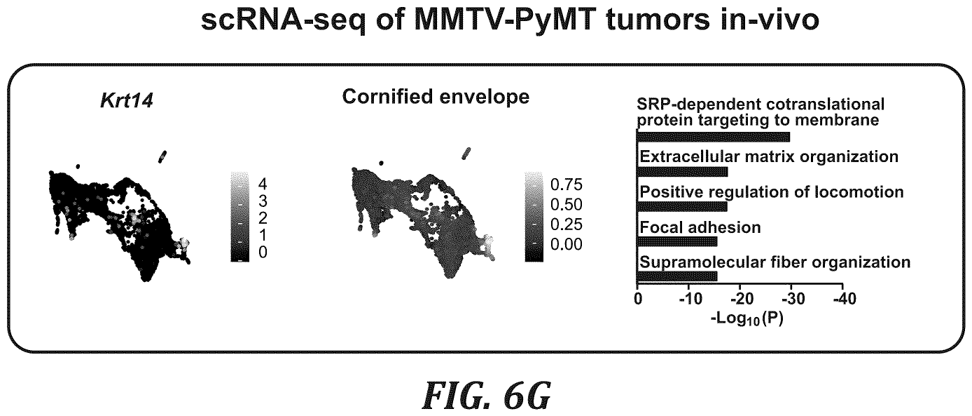

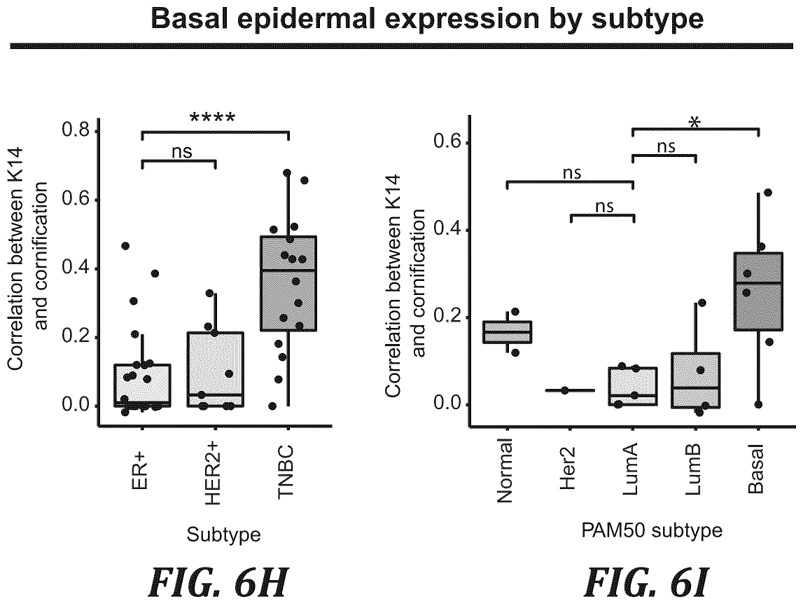

Here, single cell RNA sequencing (scRNA-seq) was used to interrogate the transcriptional dynamics of cell plasticity giving rise to basal-like epithelial cells or basal leader cells in breast cancer. Mammary tumor organoids were isolated from the Mouse Mammary Tumor Virus Polyomavirus Middle-T (MMTV-PyMT) mouse model, which forms spontaneous aggressive mammary tumors and frequently metastasizes. Transcriptionally, MMTV-PyMT tumors show similarity with human luminal B subtype breast cancers and breast tumors with low estrogen receptor expression. MMTV-PyMT tumor organoids embedded in 3D collagen rapidly formed cell protrusions that matured into multifocal invasive strands that were quantified by invasion index (FIGS. 1A, 1B, and 6A).

These events coincided with leading cells acquiring progressive expression of basal epithelial markers including cytokeratin-K14 (K14), indicating tumor cell plasticity (FIG. 1C). Three time points were selected for single cell RNA sequencing (0, 16 and 72 hours). Tumor organoids were extracted from the matrix, dissociated into single cells, encapsulated and barcoded for downstream library preparation and RNA sequencing (10× Genomics), altogether yielding RNA profiles for 9,661 cells. Each biological replicate was analyzed separately and merged together, with similar results (Methods). Uniform manifold approximation and projection (UMAP) clustered tumor cells together by time point, while bifurcating into luminal and basal-like lineages. Further, Krt14+ cells became more abundant over time, indicating that the basal leader cell program is transcriptionally regulated (FIGS. 1D, 1E, and 6B).

To better define the basal leader cell state, genes whose expression was most correlated with Krt14 transcript levels were identified next across all cells (FIG. 1F). Surprisingly, gene set enrichment analysis of transcripts positively correlated with Krt14 revealed enrichment of genes related to cornified envelope formation, epidermal differentiation, and epidermal morphogenesis, while transcripts inversely correlated with Krt14 related to brown fat differentiation and cell motility (FIGS. 1G-11, 6B, and 6C). Likewise, unsupervised annotation using the Mouse Cell Atlas annotated Krt14-high cells as neonatal skin keratinocytes (FIG. 6E), indicating that basal tumor cells transcriptionally resemble cells of the epidermis. In contrast, Krt14 transcript level were not correlated with either smooth muscle gene expression, a hallmark of mature myoepithelial cells, or mesenchymal gene expression, associated with EMT (FIGS. 1J, 1K, and 6D).

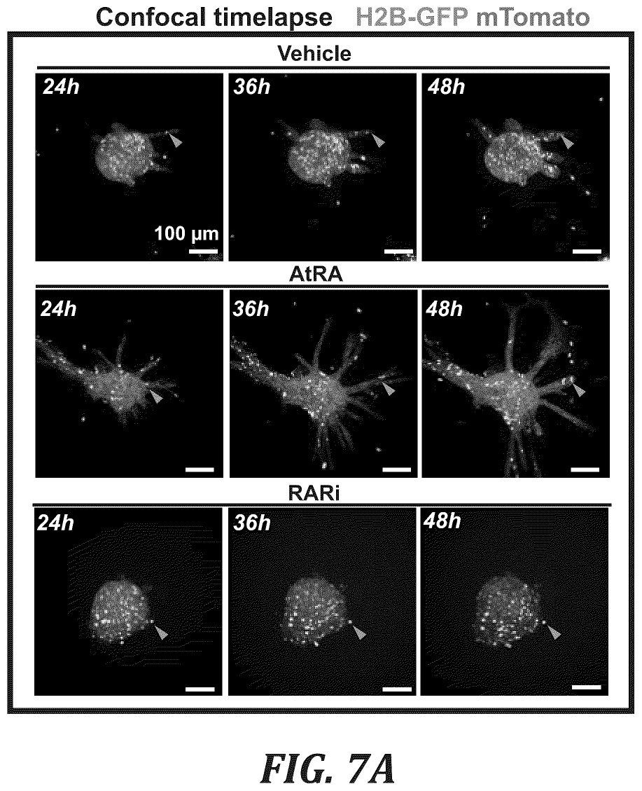

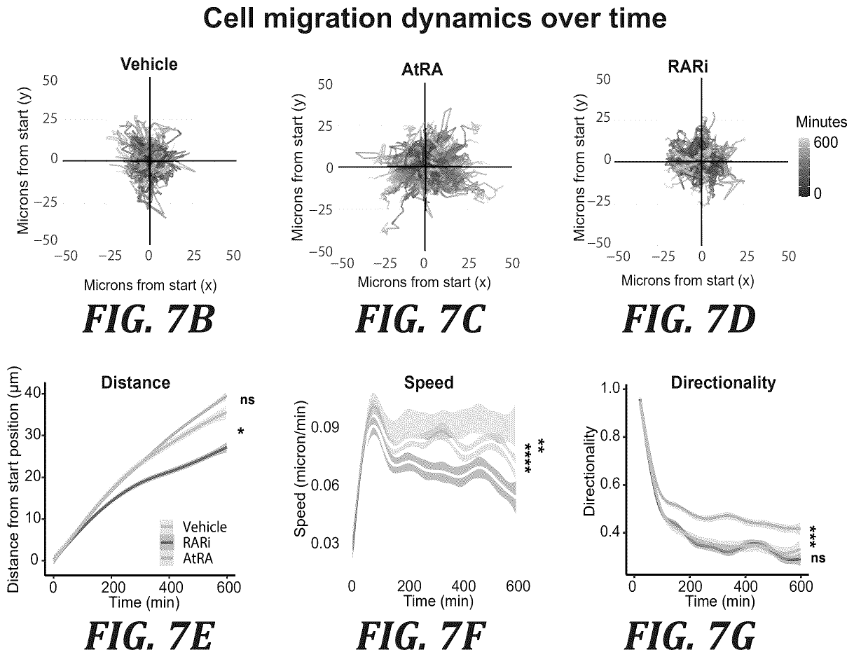

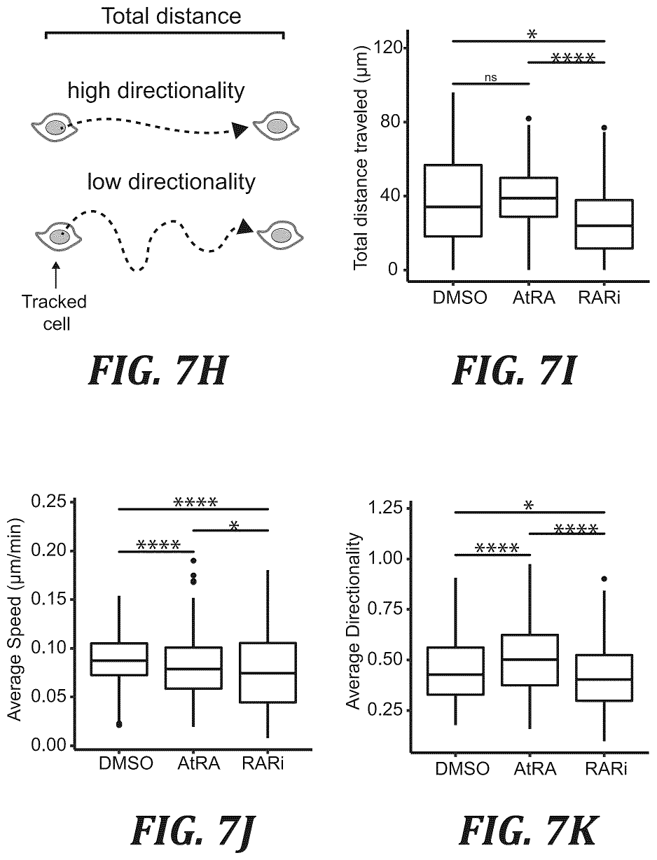

Epidermal gene programs were top enriched gene sets in basal tumor cells in scRNA-seq datasets of invading organoids tumors from the highly invasive C3 (1) Tag mouse model of basal-like breast cancer (FIGS. 1L and 6F). Extending these results further, Krt14 was found to correlate with epidermal differentiation programs in scRNA-seq of vivo tumors from three mouse models of breast cancer, MMTV-PyMT, C3 (1) Tag, and Pten-Myc (FIGS. 1L and 7G). Finally, epidermal gene set enrichment in 47 human breast patient tumors was determined from two human breast tumor scRNA-seq atlases (FIG. 1L). In both human datasets, basal tumor cells frequently co-expressed epidermal genes, particularly in tumors with triple-negative receptor status and PAM50 basal molecular subtype (FIGS. 6H and 6I).

### Example 1.1. Classical Inducers and Suppressors of Epidermal Differentiation Regulate Invasion Dynamics and Cell Viability

In skin, keratinocytes undergo a process of stratification and terminal differentiation, giving rise to a cornified rigid and crosslinked layer of dead cells at the outer surface of the epidermis. One major regulator of epidermal differentiation state is signaling by retinoic acid receptors (RARs), which are nuclear hormone receptors that bind vitamin A-derived metabolites to activate gene transcription.

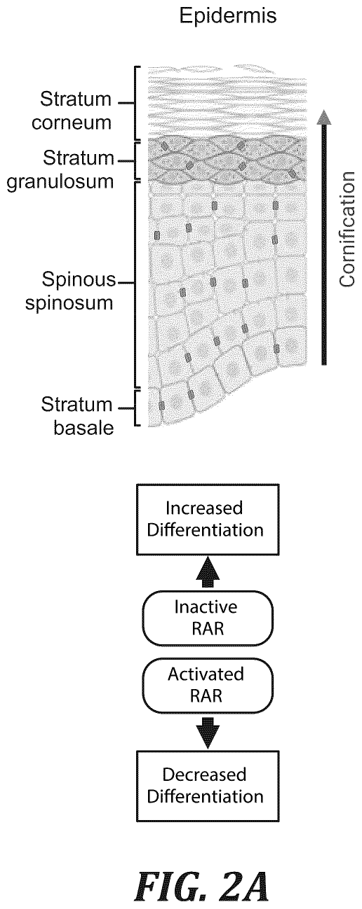

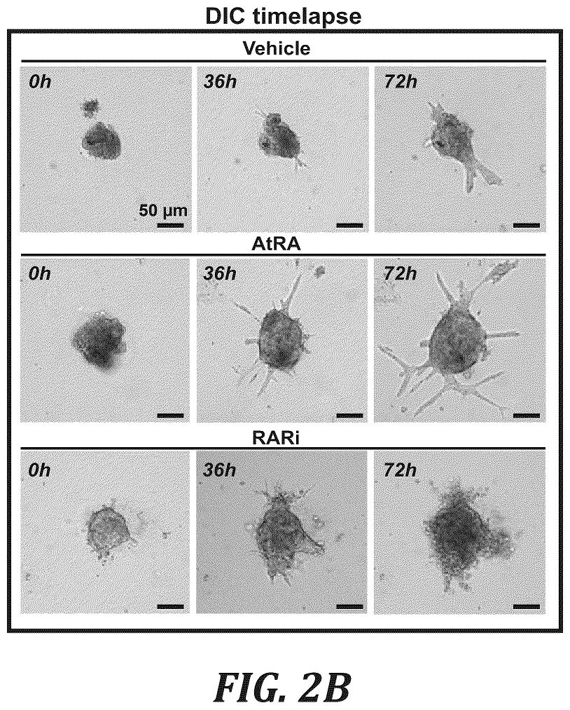

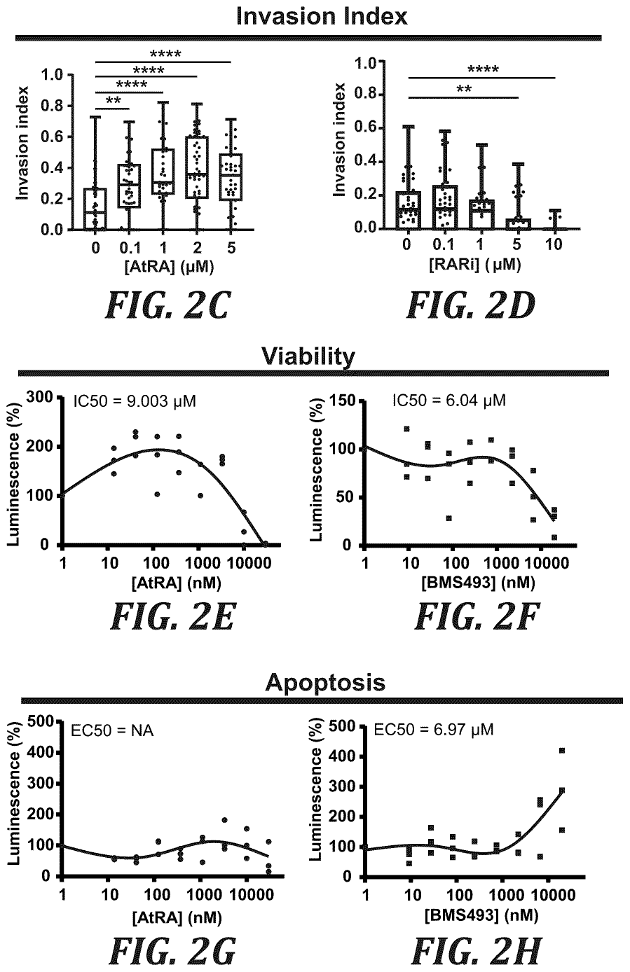

Natural and synthetic retinoic acid derivatives are used widely for a variety of dermatologic conditions to promote epidermal hyperproliferation and prevent terminal cornification. Conversely, vitamin A deficiency causes epidermal keratinocytes to undergo excessive cornification, which is associated with glandular-to-squamous metaplasia. Thus, vitamin A, through its metabolite, all-trans retinoic acid (AtRA), activates RAR signaling and opposes epidermal differentiation, while deficiency of vitamin A reduces RAR signaling to promote differentiation (FIG. 2A).

Given that basal breast tumor cells resemble an epidermal-like cell state, the next question asked was how basal breast tumor cells would respond to regulators of epidermal differentiation. The effects of retinoic acid signaling on MMTV-PyMT tumor organoid dynamics was examined using AtRA to induce retinoic acid receptor (RAR) activity, and the retinoic acid receptor inhibitor, pan-RAR reverse agonist BMS493 (RARi) to mimic retinoic acid deficiency (FIG. 2B), at typical dose ranges used to modulate keratin expression. Within 72 hours, both agents elicited striking responses. AtRA-treated organoids at doses ranging from 0.1 to 5 μM produced greater numbers of collective invasion strands, that were also longer and narrower, when compared to control (FIGS. 2C and 2D). In contrast, organoids treated with RARi had little to no collective strands at end point, and organoids dissociated into rounded-up single cells that were stationary (FIGS. 2C and 2E).

Next, to quantify individual cell dynamics, tumor organoids were transduced with a nuclear H2B-GFP reporter, and individual cells were tracked by confocal microscopy (FIG. 7A). Surprisingly, single cell migration tracking revealed that AtRA did not increase net individual cell speed. Rather, AtRA increased cell persistence indicating improved net efficiency of directional collective migration (FIGS. 7B-7K). RARi treatment, however, led to decreased distance traveled and speed of individual cells, with evidence of nuclear blebbing and fragmentation (FIGS. 7B-7K).

Since the observations in RARi treated organoids were consistent with cell death, viability and apoptosis of treated organoids were assessed using CellTiter-Glo® and Caspase-Glo® 3/7 assays, respectively. The results indicated that RARi treatment reduced viability and increased caspase activity in a dose dependent manner, with an IC50 of 6.0 μM (FIGS. 2G and 2H). Interestingly, response curves for AtRA were biphasic, with increased viability and reduced caspase activity at lower doses, followed by reduced viability and increased caspase activity at high doses with IC50 of 9.0 μM (FIGS. 2F and 2H). These results indicate that AtRA treated organoids, specifically at low doses, are less prone to apoptosis and more highly proliferative or metabolically active.

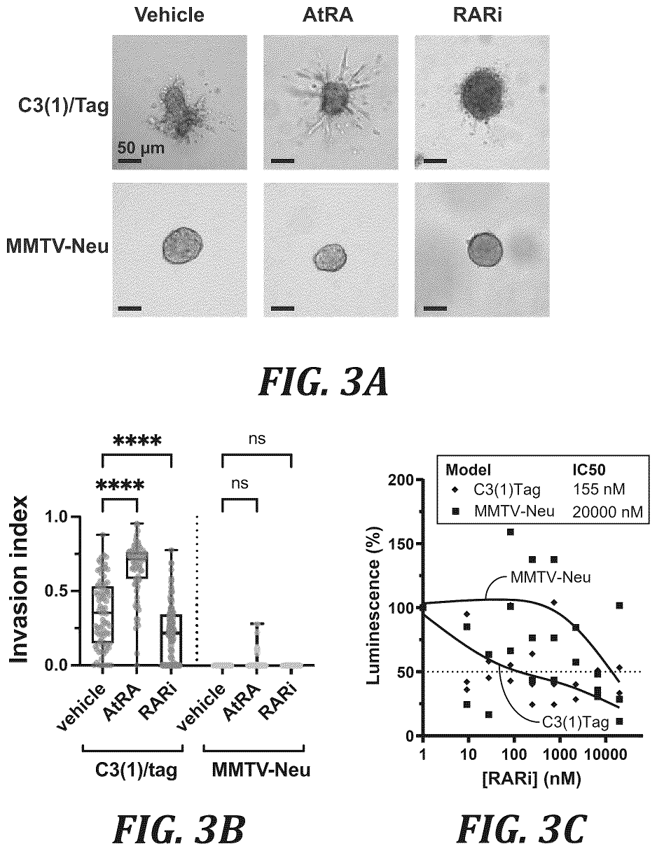

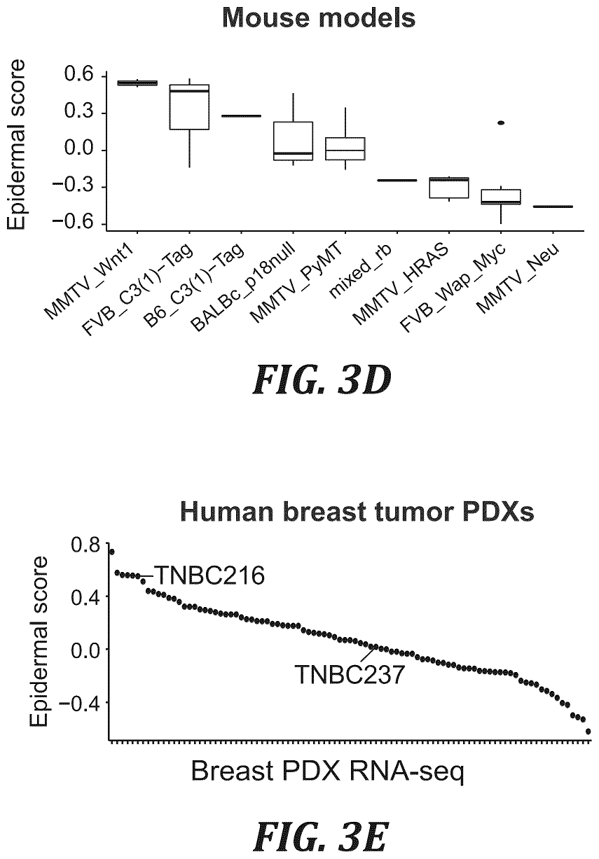

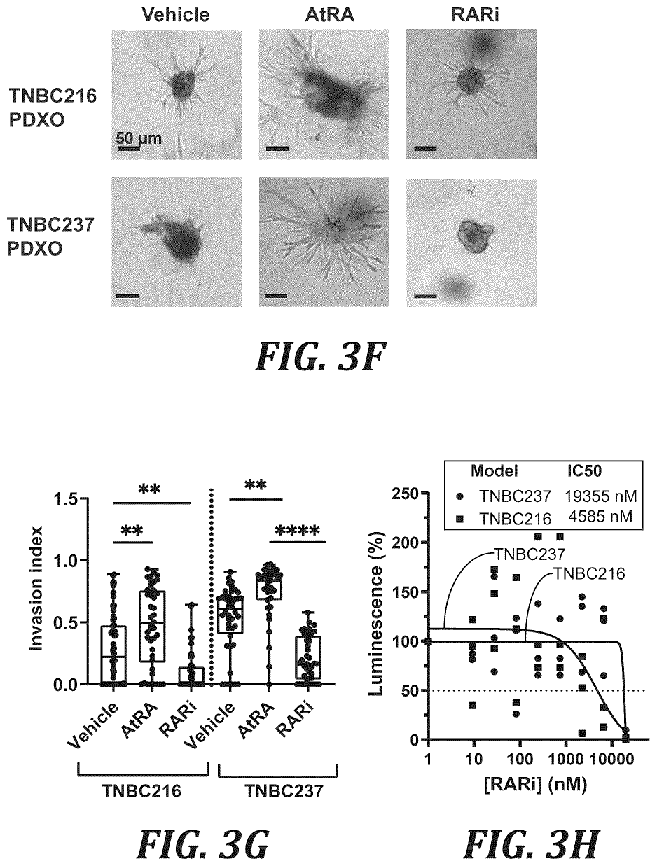

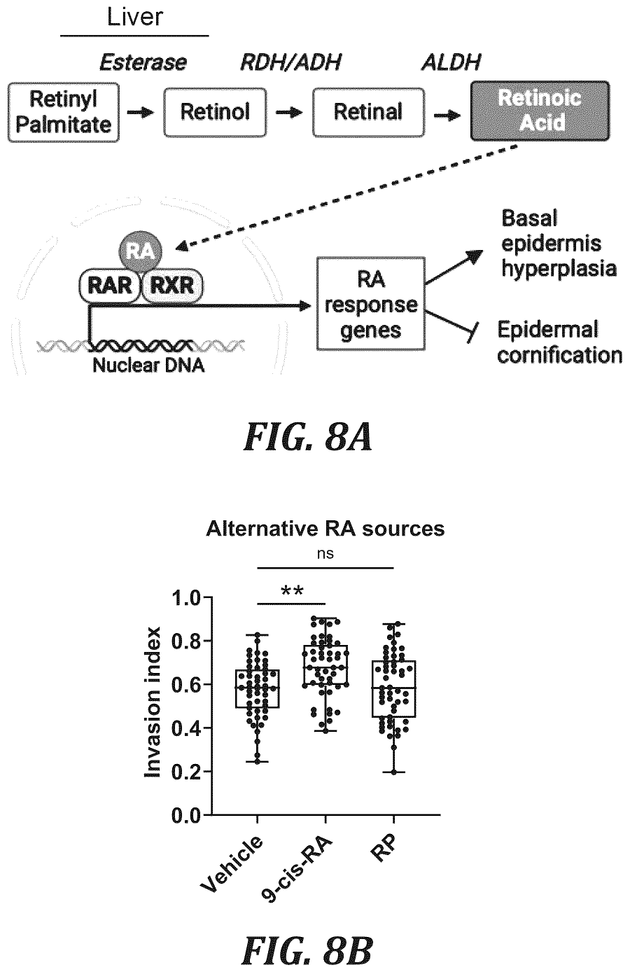

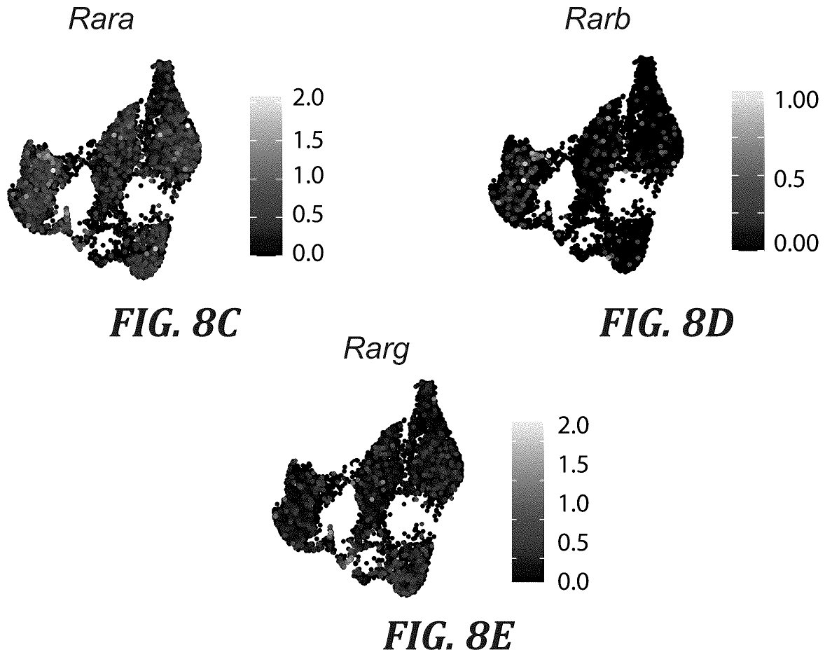

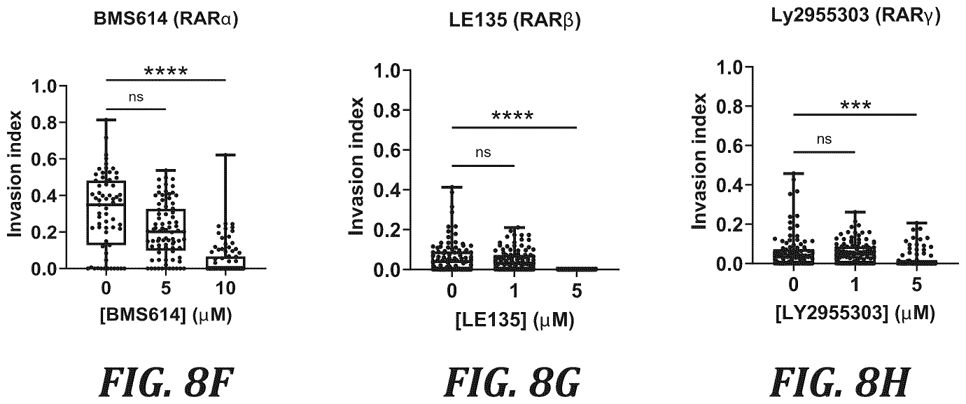

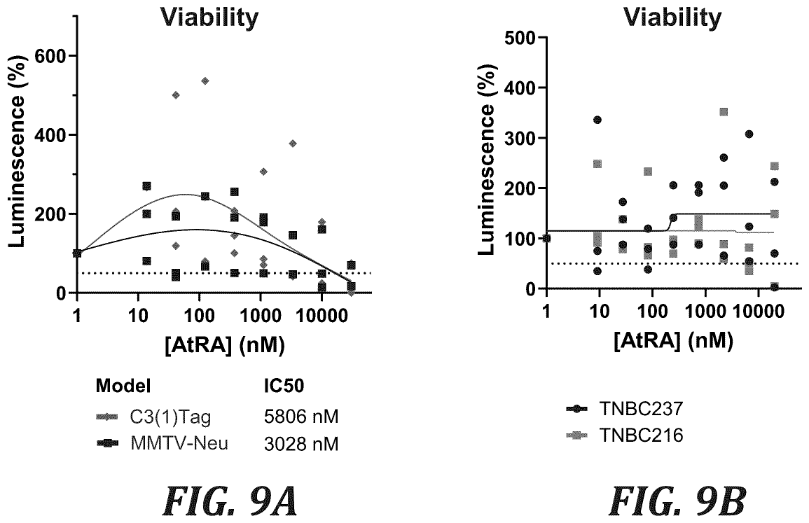

To confirm that retinoic acid receptor activity regulates invasion, the tested drug panel was expanded to include two additional retinoic acid sources: 9-cis-retinoic acid (9-cis-RA) and retinyl palmitate. Increased invasion was observed upon treatment with 9-cis-RA, which can bind and activate RARs directly, but as expected, no significant difference in invasion when organoids were exposed to retinyl palmitate, which is the storage form, normally enzymatic processed into retinoic acid by hepatic stellate cells (FIGS. 8A and 8B). In the invasion scRNA-seq dataset described herein, RAR transcript expression was broadly uniform throughout the uMAP (FIGS. 9C-9E). When treated with specific inhibitors for RARα, RARβ, and RARγ isoforms using BMS614, LE135, and LY2955303, respectively, tumor organoids had reduced invasion, indicating that RAR nuclear receptor activation, via one or more isoforms, is necessary for repression of invasion and tumor cell survival (FIGS. 3F-3H).

### Example 1.2. Cornification Signature is Predictive of AtRA Induced Invasion and Proliferation, and RARi Induced Cell Death in Mouse and Human Breast Tumor Organoid Models

Given the robust effects of retinoic acid signaling perturbations in the MMTV-PyMT organoid model, the generality of this response was determined next. Two models were chosen for further study: C3 (1)/tag, a mouse model of basal breast cancer, and MMTV-Neu a model of luminal breast cancer. By timelapse imaging, C3 (1)/Tag organoids treated with AtRA displayed a pronounced increase in invasion, including in some cases shattering of organoids to individually highly migratory cells (FIGS. 3A and 3B). By CTG assay, C3 (1) tags showed marked loss of cell viability in response to RARi with an IC50 of 155 nM (FIG. 3C). In contrast, MMTV-Neu, the luminal model, had minimal responses to either AtRA or RARi (FIGS. 3A-3C). Given the marked differences between models, it was then hypothesized that these models might have differences in epidermal gene expression. To answer this question, common mouse models of breast cancer were ranked by the epidermal cornification signature identified from scRNA-seq (FIG. 3D). This demonstrated that C3 (1)/Tag organoids were among top scoring model for epidermal signature, whereas MMTV-Neu scored the lowest.

To extend these results further, the next question asked was whether epidermal cornification signature was predictive of AtRA and RARi response in human breast tumors. Human patient derived breast tumor organoids with RNA-seq data were ranked by cornification score (FIG. 3E). Two TNBC models were tested, TNBC216 and TNCB237, to represent high and moderate epidermal cornification scores, respectively. Each model was treated with AtRA and RARi and invasion was determined by collagen assay and CellTiter-Glo®. It was found that invasion rates in both tumor organoid lines were responsive to AtRA and BMS493 (FIGS. 3F, 3G, 9A, and 9B). In a similar manner to the mouse model organoids, TNBC216—which had the higher cornification score—was more sensitive to RARi than TNBC237 (FIG. 3H). Taken together, these data indicate that cornification epidermal transcriptional signature is predictive of AtRA induced invasion and RAR inhibitor induced cell death in both mouse and human breast cancer organoid models.

### Example 1.3. RAR Activation in Breast Tumor Organoids-Limits Epidermal Differentiation and Keratinization

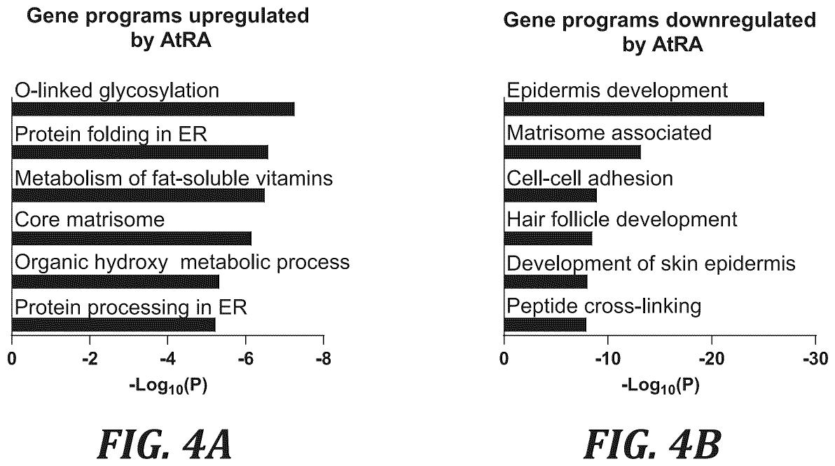

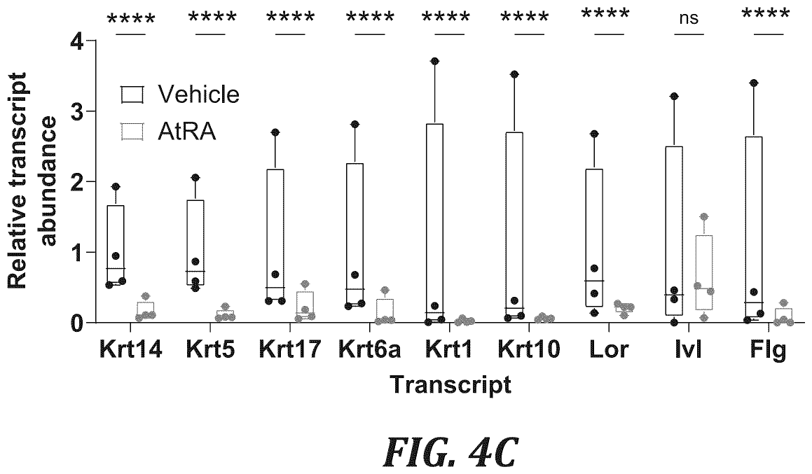

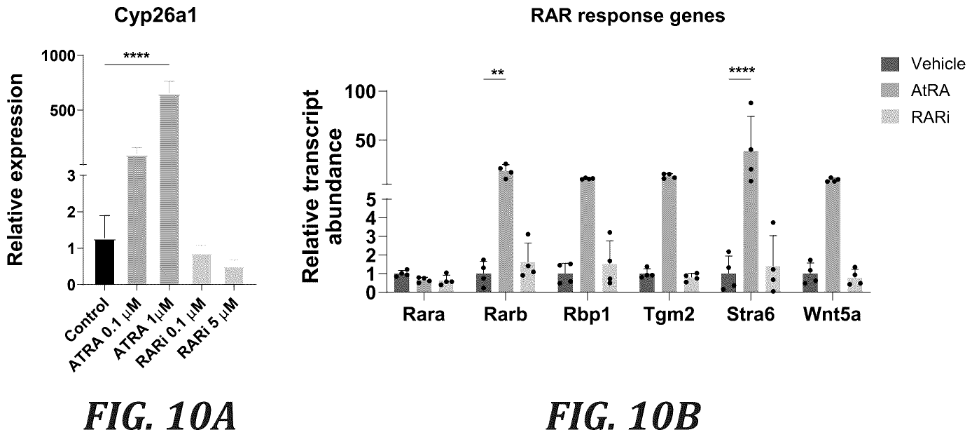

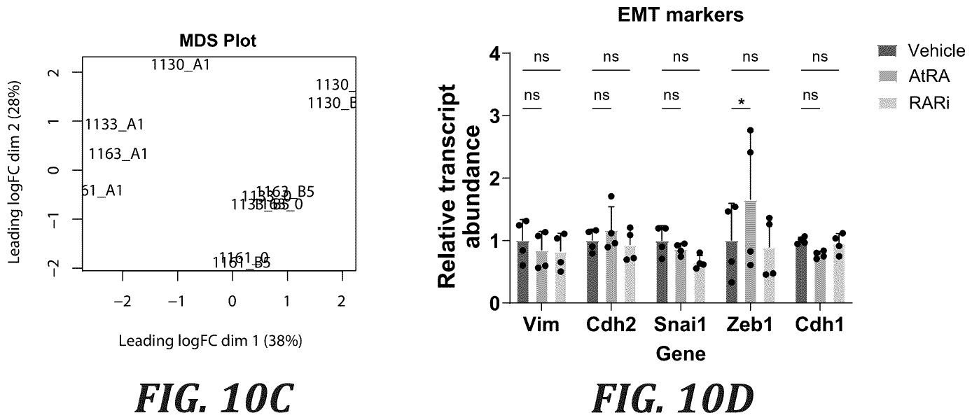

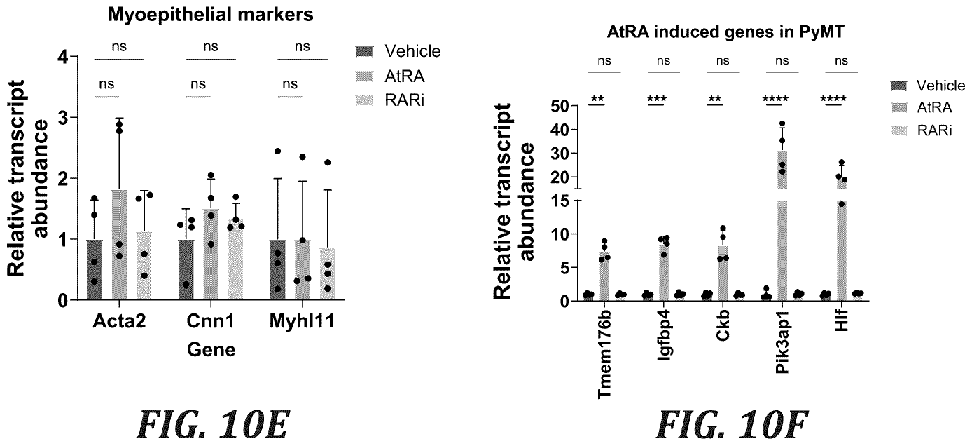

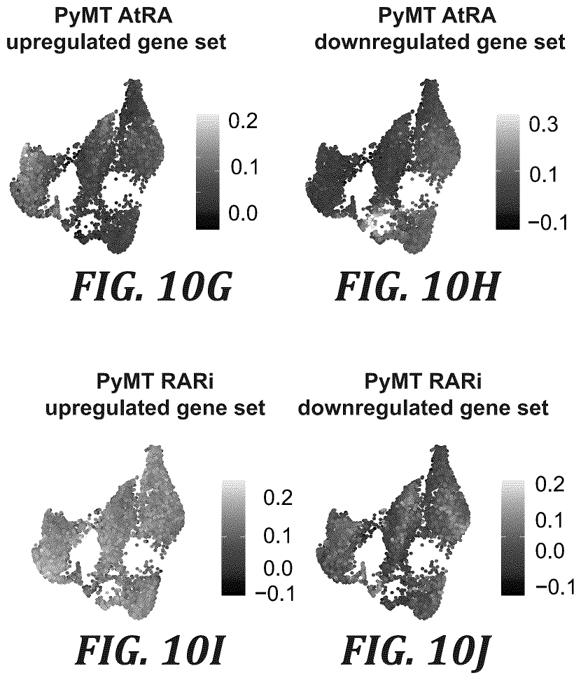

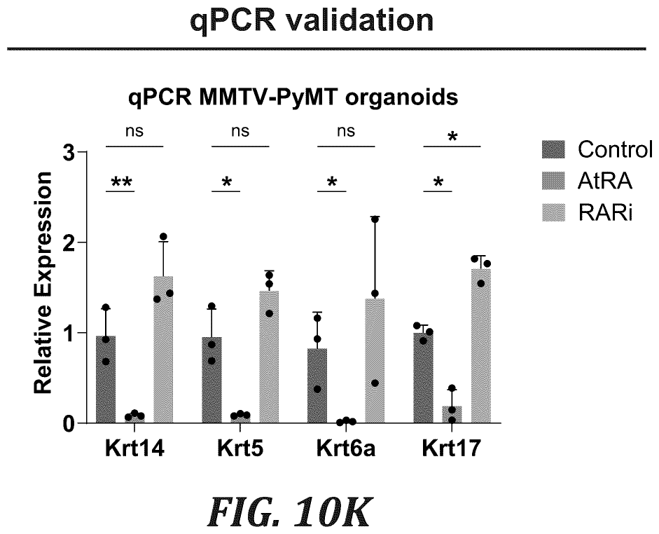

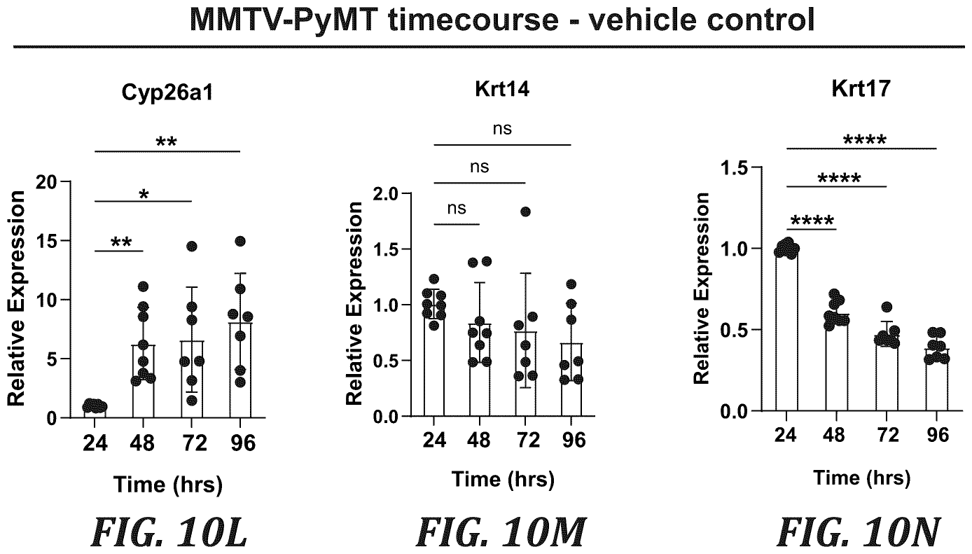

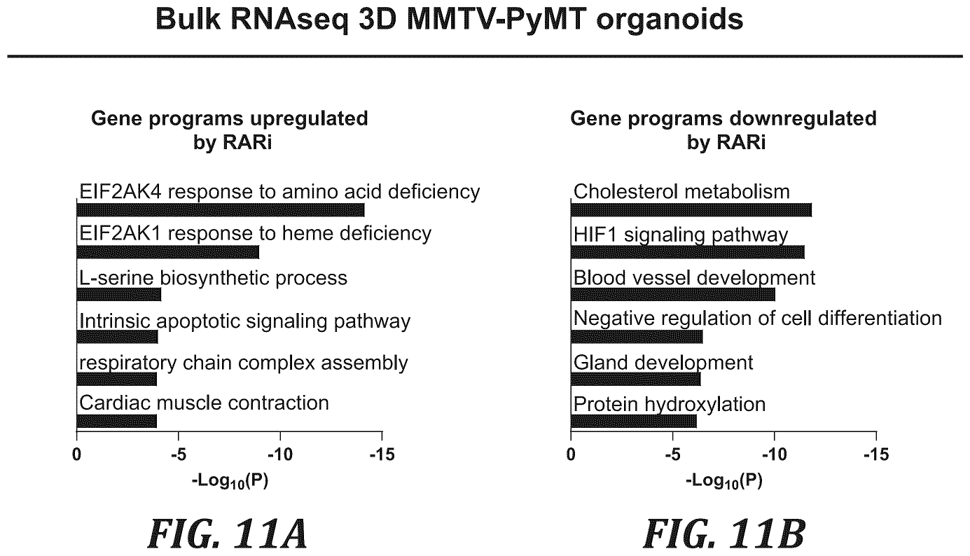

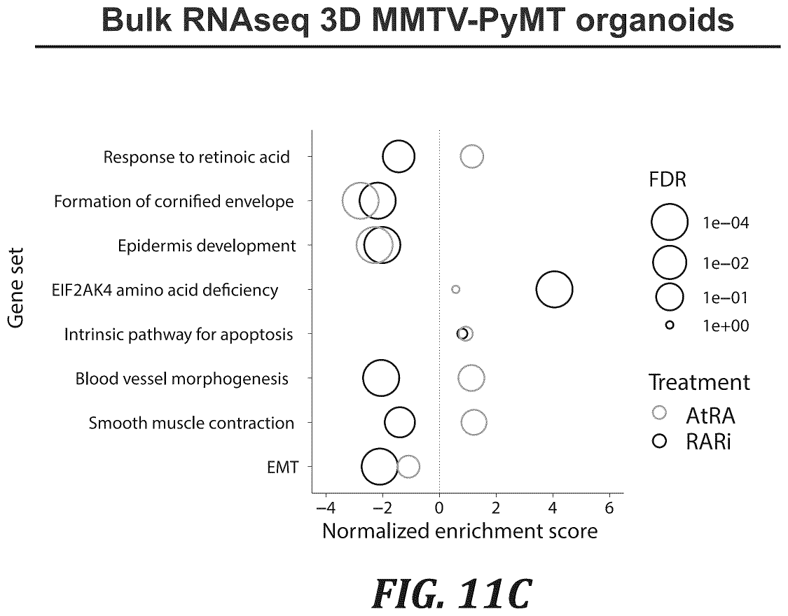

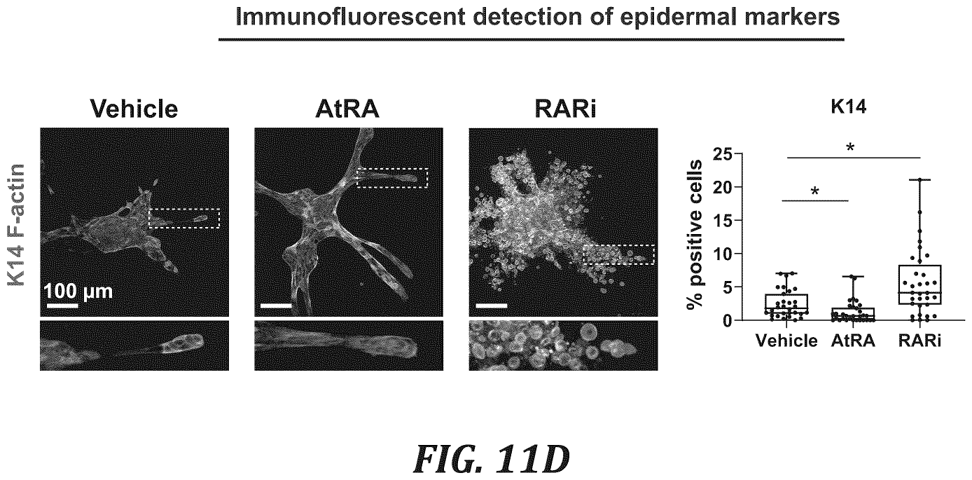

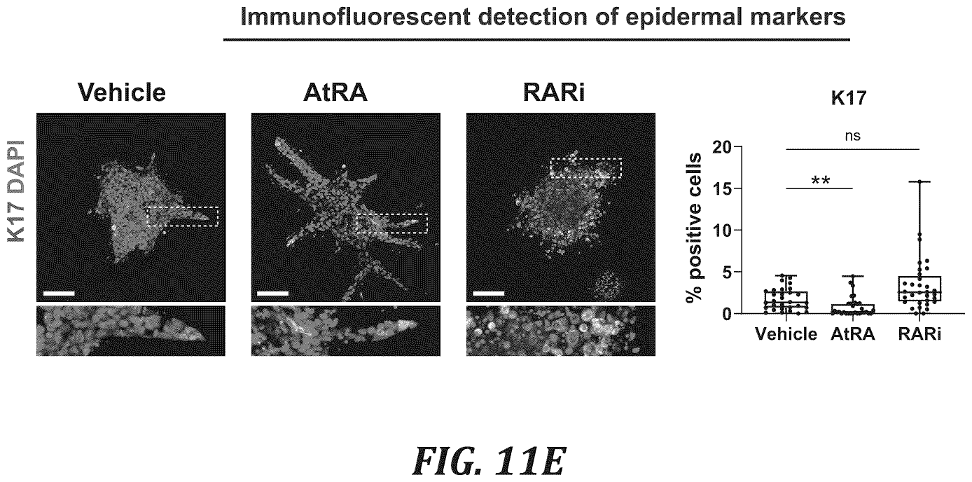

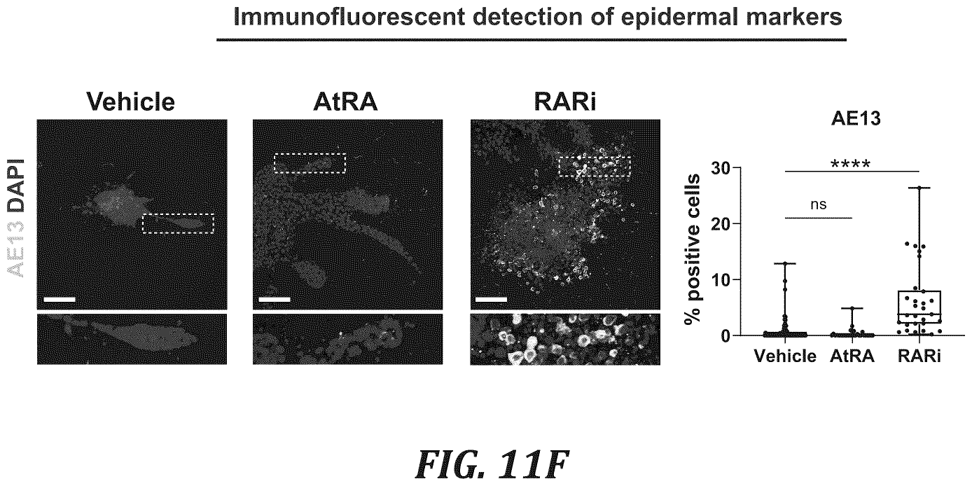

Given the specificity of RAR signaling in epidermal-like breast cancer models, the next question was how RAR signaling modulates gene expression on a molecular level. To address this question, RNA-seq was performed on AtRA and RARi treated PyMT organoids. As expected, canonical retinoic acid response gene Cyp26a1 and RAR target genes were strongly induced by AtRA treatment and reduced by RARi treatment in a dose dependent manner (FIGS. 10A and 10B). By PCA plot, AtRA treatment induced large-scale changes in gene expression, whereas RARi induced more modest changes, likely due to widespread cell death associated with RARi (FIG. 10C). Top gene programs induced by AtRA included genes involved in O-linked glycosylation, ER protein processing, and fat-soluble vitamin/retinol metabolism (FIGS. 4A and 10F). Notably, tumor cells retained epithelial gene expression and did not induce markers of canonical EMT or myoepithelial cells (FIGS. 10D and 10E). Top gene programs suppressed by AtRA included genes involved in epidermis development, barrier establishment, and hair follicle development (FIG. 4B). These gene sets localized to invasive cells in the single-cell RNAseq data described herein, whereas the AtRA upregulated gene set localized to early timepoint cells (FIGS. 10G-10J). Consistent with the effects of AtRA in cultured keratinocytes, genes associated with epidermal terminal differentiation were uniformly downregulated by AtRA (FIG. 4C), which were further confirmed on an RNA level by qPCR for Krt14, Krt5, and Krt6a (FIG. 10K), and by immunofluorescence for K14 and K17 (FIGS. 11D and 11E). Conversely, RARi treated organoids displayed increased expression of basal keratins K14 and K17 (FIGS. 11D and 11E), and to a lesser extent AE13, an antibody reactive with hair keratins (FIG. 11F). Taken together, these observations reveal that retinoic acid signaling induces a non-EMT migratory program and limits epidermal differentiation and keratinization.

### Example 1.4. RAR Signaling Antagonism Markedly Inhibits Tumor Growth and Metastatic Progression In Vivo

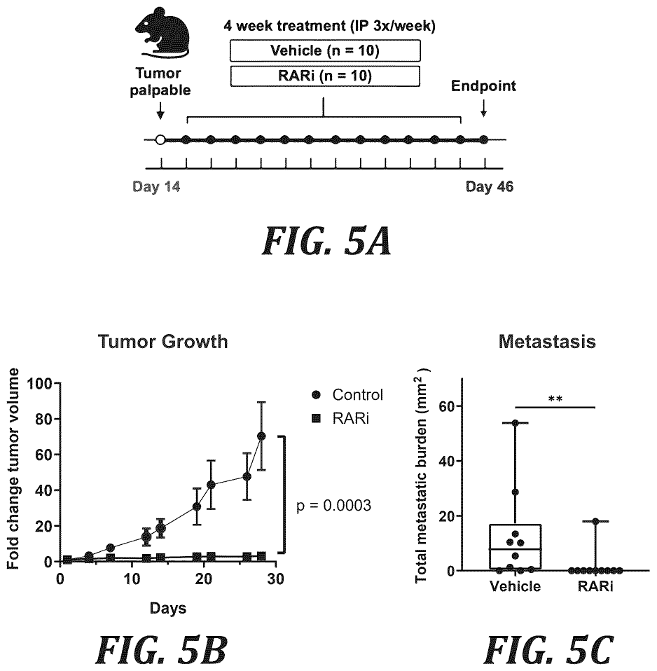

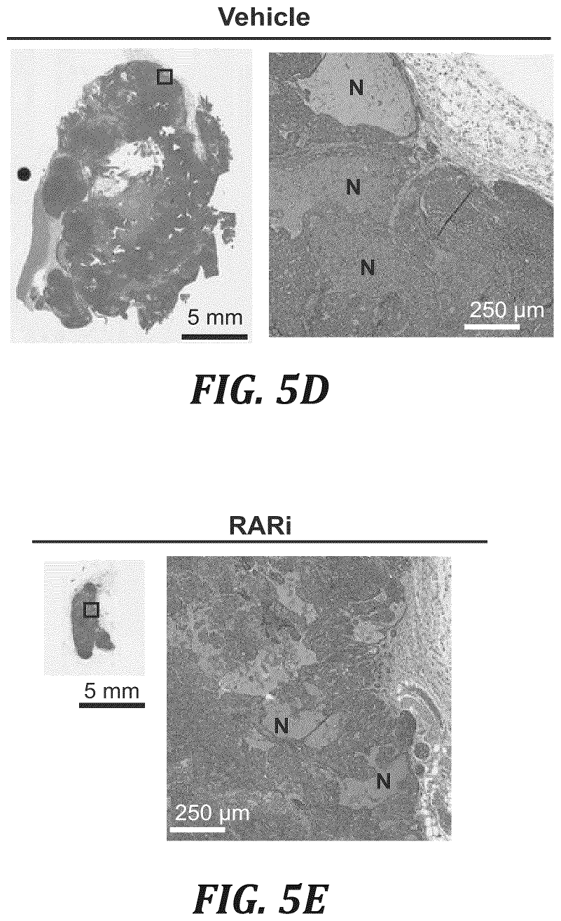

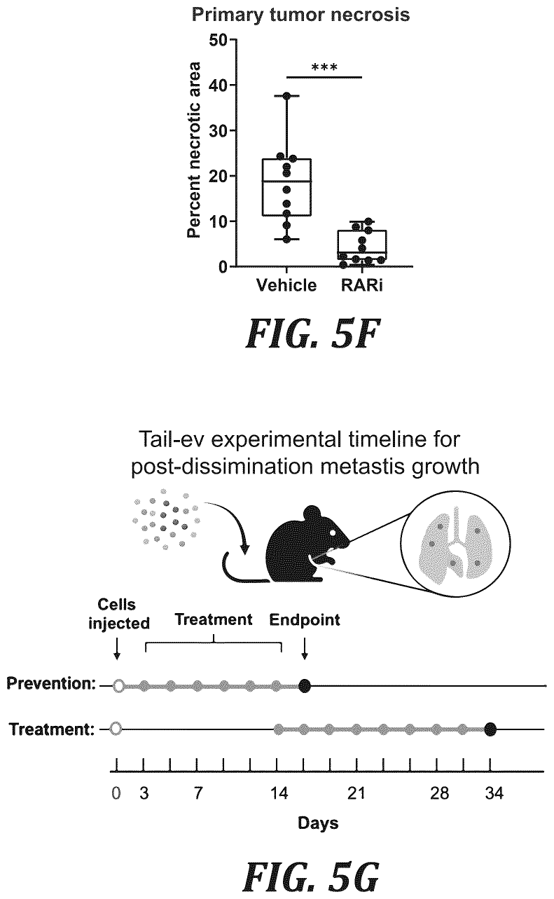

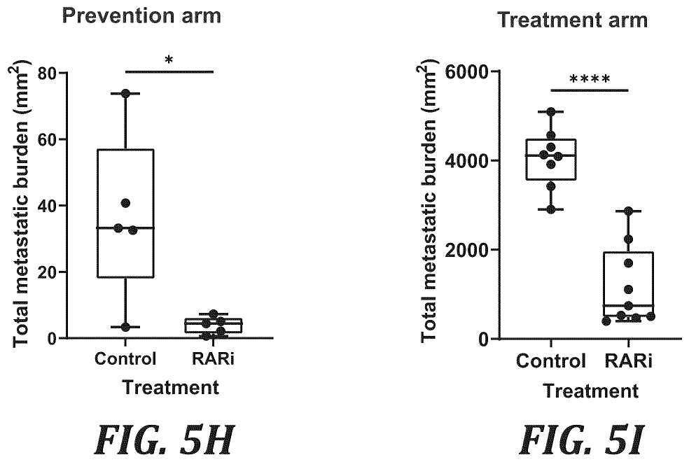

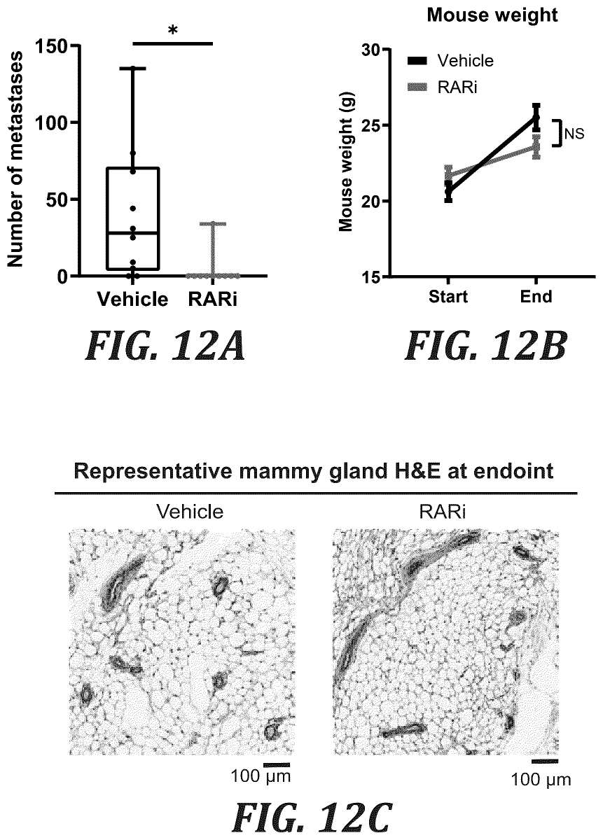

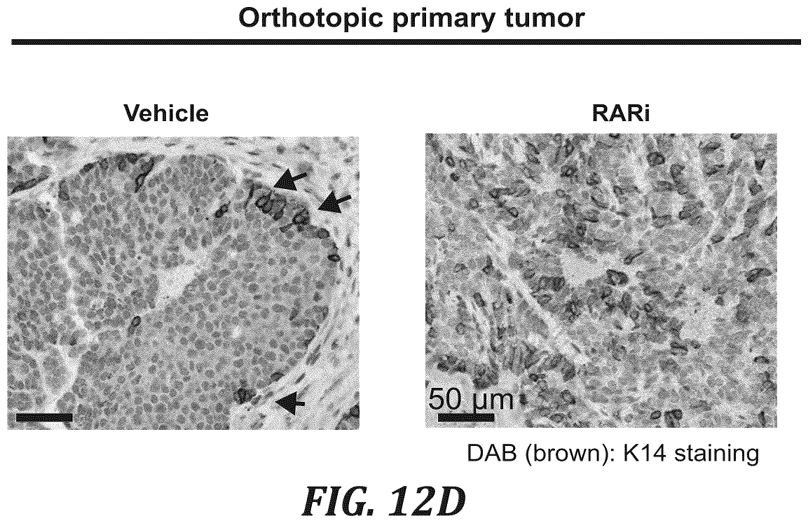

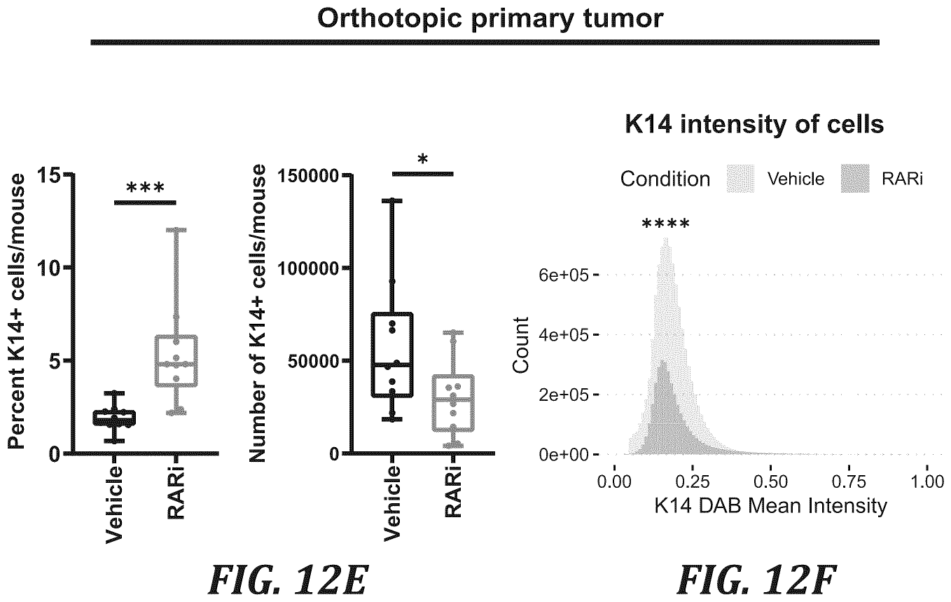

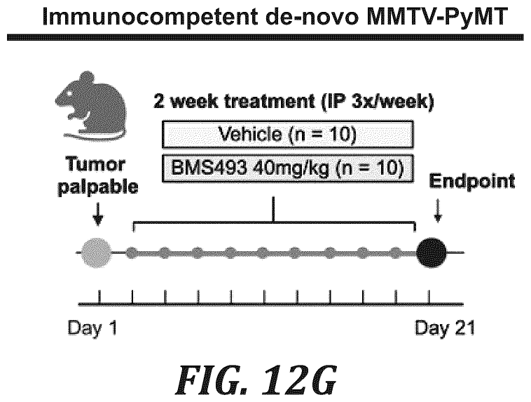

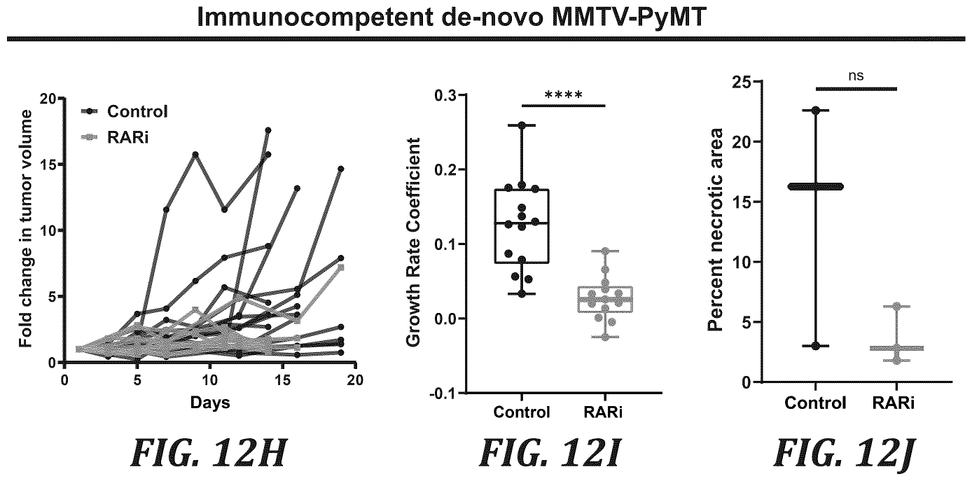

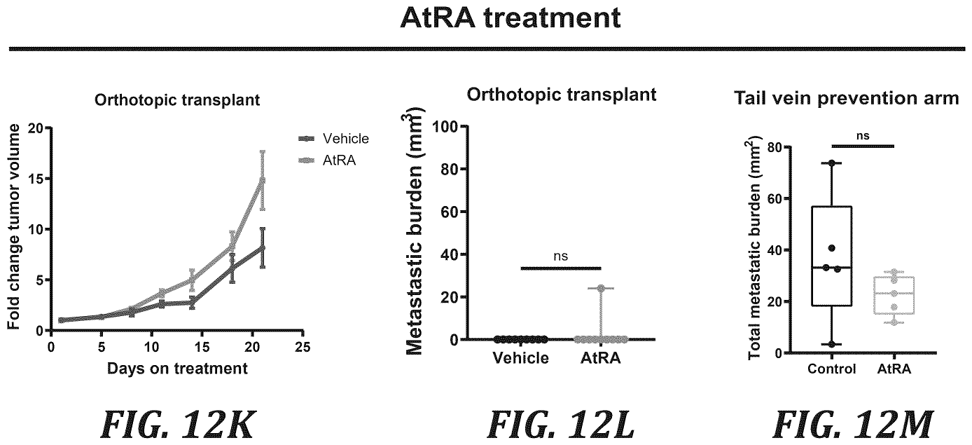

Next, the effect of suppressing retinoic acid signaling in vivo was tested. To answer this question, MMTV-PyMT tumor organoids were transplanted into immunocompromised NSG mice, and then upon reaching 200 mm² in size, treatment began with a 4-week course of vehicle or RARi (BMS493) 40 mg/kg given intraperitoneally (i.p.) three times weekly. In vivo, shutting off RAR signaling markedly suppressed tumor outgrowth and prevented the formation of spontaneous metastasis (FIGS. 5A-5C and 12A). Importantly, RARi was well tolerated, with no differences in weight between treatment conditions and no marked differences in histology of contralateral normal mammary glands (FIGS. 12B and 12C). By H&E, tumor histology was consistent with poorly differentiated carcinoma in both control and RARi treated tumors. However, RARi treated tumors were much smaller and had markedly fewer areas of necrosis, a histologic feature associated with metastatic dissemination (FIGS. 5D-5F). Given the effect of RARi on basal keratin expression in organoids, immmunohistochemical staining of tumors for K14 was performed next. This revealed a change in cellular organization and composition. In control tumors, K14+ cells were located at the basal edges of tumors, while in RARi tumors, K14+ cells were spread throughout the tumor (FIG. 12D). Numerically, total K14+ cells were less abundant but accounted for relatively higher percentage of cells (FIG. 12E), and mean K14 expression was decreased on individual cell basis with RARi treatment (FIG. 12F).

Extending these results further, a preclinical study of RARi in immunocompetent MMTV-PyMT mice harboring spontaneous tumors was performed next. In the immunocompetent setting, RARi led to symptoms of xerophthalmia, a common side effect of vitamin A deficiency, precluding long-term treatment. Notably, short term RARi also suppressed tumor growth and necrosis in immunocompetent MMTV-PyMT mice (FIGS. 12G-12J).

Given the strong impact of RARi on primary tumors, effects of RARi on metastatic colonization were defined next. Fluorescently tagged MMTV-PyMT clusters were injected into the tail veins of NSG mice, and after 3 days, treatment with RARi was initiated for 2 weeks and thereafter, metastatic burden was assessed. RARi markedly reduced the number and size of metastases. Next, to model the impact on already established metastases, MMTV-PyMT tumor cells were injected into tail veins of NSG mice, and thereafter the metastases were allowed to grow for two weeks before initiating a 3-week course of RARi. Likewise, in the already-established metastasis setting, RARi treatment markedly reduced metastatic tumor burden.

Complementary orthotopic transplant and experimental metastasis studies were conducted treating mice with AtRA at a dose chosen in the range given to acute promyelocytic leukemia patients (12 mg/kg, 3x weekly). In these experiments, no effect of AtRA on tumor growth or metastasis was observed in these mice fed a vitamin A sufficient diet (FIGS. 12K-12M). In total, these data show that RAR signaling suppression, not activation, markedly inhibits tumor growth, metastatic seeding and metastatic progression in an aggressive mouse model of breast cancer.

### Methods

### Animal Models

All mice were maintained under specific pathogen-free conditions, and experiments conformed to the guidelines as approved by the Institutional Animal Care and Use Committee of Fred Hutchinson Cancer Center (FHCC). FVB and MMTV-PyMT (FVB/N-Tg (MMTV-PyVT) 634Mul/J) were obtained from Jackson Labs. NSG (NOD skid gamma) mice were bred by Fred Hutchinson Cancer Center shared resources.

### Mouse Organoid Preparation and Culture

Organoids were isolated from MMTV-PyMT, C3 (1) TAg, or MMTV-Neu mouse mammary tumors as previously described (Nguyen-Ngoc et al., 2015, Wrenn et al., 2020). Mice were harvested as the largest tumor neared 1-2 cm in diameter. Mammary tumors were dissected, minced with a scalpel, and then digested in a collagenase-trypsin solution for 30-60 minutes shaking at an angle at 100-150 rpm at 37° C. The digestion solution contained 2 mg/mL collagenase (Sigma-Aldrich® C2139), 2 mg/mL trypsin (GIBCO™ 27250-018), 5% fetal bovine serum, 5 μg/mL human insulin (Sigma-Aldrich® 19278), 50 μg/mL gentamicin (GIBCO™ 15750-060) in 20 mL of DMEM/F12. After digestion, larger tumor fragments were removed by allowing them to settle in the tube, while the remaining solution was moved to a new tube. Single cells and debris were removed from the smaller fragment solution by centrifuging for 4 seconds at 450 g to isolate multicellular organoids, then resuspended in 10 mL DMEM/F12. This was repeated for a total of 4 washes. For suspension culture, clusters were cultured in non-adherent dishes in organoid media: DMEM/F12 (Thermo Fisher® 10569044), 1% Penicillin-Streptomycin (Sigma-Aldrich® P4333), 1% ITS-X (Thermo Fisher® 51500056), 220 ng/ml hFGF-Basic (PeproTech® 100-18C), and 2% (v/v) reduced growth factor Matrigel®. Tips and tubes used to handle organoids were first coated in 2.5% BSA in DPBS to prevent loss of material.

### Human Organoid Preparation

TNBC216 (J000106527/BCM-3469) and TNBC237 (J000106528/BCM-2147) patient derived xenografts (PDXs) were obtained from Jackson labs and maintained in mice by the preclinical modeling core lab at Fred Hutch Cancer Center. After tumor collection, tumors were processed into organoids using the same methodology as used for mouse tumor organoids, above.

All PDX organoids were cultured in suspension in media previously published 470 (cite) containing Advanced DMEM/F12, 5% FBS, 1×HEPES, 1× Glutamax, 1 μg/mL hydrocortisone, 0.1 μg/mL gentamicin, 10 ng/mL human EGF, 10 μM Y-27632, 100 ng/ml FGF2, 1 mM NAC and 5% (v/v) reduced growth factor Matrigel®.

### Preparation of Rat-Tail Collagen

The collagen preparation protocol described herein was developed with guidance from Dr. Kayla Bayless (Bayless et al., 2009), and adapted from Bornstein et al, 1958, and Rajan et al., 2006. Freeze dried rat tails were purchased from Pel-Freez® and stored at −80° C. For collagen preps, two tails were thawed at room temperature in 70% ethanol. Sections of tail were progressively removed, pulling the collagen fibers from the tail. The collagen fibers were cleaned in water, then cut into small pieces and dissolved in 0.1% acetic acid (MilliporeSigma® A6283, dissolved in sterile ultra-pure water) for 8 days at 4° C., occasionally centrifuging the collagen and collecting the supernatant. All collected supernatant was split into 50 mL conical tubes and frozen in dry ice. The collagen was then lyophilized with the assistance of the Proteomics and Metabolomics Core at the Fred Hutchinson Cancer Center. The dried collagen was then weighed and reconstituted at a concentration of 4.5 mg/mL in 0.1% acetic acid.

### 3D Collagen Culture of Organoids or Spheroids for Invasion

Organoids were embedded in 1.5 mg/mL collagen gels, which were prepared from the 4.5 mg/mL rat-tail collagen described above using protocols previously published (Cheung et al, 2013). 4.5 mg/mL collagen was diluted to 1.8 mg/mL in 0.1% acetic acid. This was then mixed with 10×DMEM, 1 N NaOH, and ultrapure water at the following ratio: 1 mL collagen (1.8 mg/ml): 100 μL 10×DMEM: 32 μL NaOH: 66.66 μL H2O. This mixture was allowed to polymerize at 4° C. and was then mixed with organoids at a concentration of ~180 organoids/100 μL. Glass-bottom 24 well plates were prepared by plating 40 μL of collagen (without organoids) in the center of each well to be used. The plate was placed at 37° C. for 5-10 minutes to allow the collagen disks to polymerize, after which 100 μL of the collagen/organoid mixture was plated on top as a dome. This was allowed to polymerize for an additional 45-60 minutes at 37° C. After complete polymerization, 1 mL of organoid media (described in organoid culture section, without Matrigel®) was added to each well.

### In-Culture Drug Studies

BMS493 (MedChemExpress®, HY-108529) and all-trans retinoic 503 acid (AtRA, StemCell Technologies™ 72262) were reconstituted in DMSO. For treatment, drugs were further diluted in DMSO to achieve a 1000x concentration of each desired dose, which was then diluted (1:1000) in the appropriate media for the cells being treated. This was then added to the media of 3D collagen cell cultures. Since retinoids are light-sensitive and unstable, treated cells were kept from bright light exposure and the drug containing media was changed every 48 hours.

### 3D Culture DIC Time-Lapse Imaging

Differential interference contrast (DIC) time-lapse images were captured hourly using a Leica® SPE microscope at 10× magnification, while temperature was maintained at 37° C. and CO₂ at 5%.

### Invasion Index

After 3 days of invasion in collagen, organoids were fixed in 4% PFA in PBS for 12-15 minutes at room temperature and stored at 4° C. 10× magnification DIC images of fixed organoids were acquired on a Leica® SPE microscope. Using Fiji™, the area of the total organoid and the center of the organoid were outlined and measured. The center area was subtracted from the total area, leaving the invasive area of the organoid. This was divided by the total area to calculate the fraction of the organoid that was invasive, or the invasion index.

### Time-Lapse Fluorescent Single Cell Tracking

For single cell tracking, membrane tomato (mTmG) MMTV-PyMT organoids were sparsely transduced with a H2B-GFP lentiviral vector generously provided by the Paddison Lab at FHCC. Fluorescent time lapse images were acquired every 20 minutes using an Andor™ CSU-W confocal spinning disk on a Leica® DMi8 inverted microscope at 20× magnification. Temperature was maintained at 37° C. and CO₂ at 5%. Z-stacks were max-projected and stabilized in Image-J. CellProfiler was used to track nuclei.

### Viability and Caspase Assays

50 μL of collagen containing 30 organoids were plated into each well of opaque white 96-well plates following the 3d collagen protocol detailed above. After plating, cells were incubated with 95 μL of Matrigel-free organoid media. In a separate round bottom 96-well plate, compounds were serially diluted 3-fold from max concentration in media to achieve a 20× drug concentration. 5 μL of 20× drug was then added to each well. Maximum concentration of BMS493 was 20 μM while maximum concentration of AtRA was 30 μM. A no-cell control, vehicle control, and puromycin control were included. All conditions were plated in triplicate. After 72 hours, CellTiter-Glo® 3D (Promega®) and Caspase-Glo® 3D 3/7 (Promega®) assay kits were used according to instructions and luminescence was read on a BioTek Synergy H1TM Hybrid Multi-Mode Reader. After reading, the no-cell control background was subtracted from all wells. Each well reading was normalized to the average luminescent signal of the vehicle control and then were averaged, representing one replicate. Vehicle control and puromycin control were used as upper and lower constraints, respectively, for IC50 calculation.

### 3D Culture Immunofluorescence

At the appropriate end point for 3D collagen cell culture experiments, media was removed and PBS was added for 5-10 minutes. Afterwards, 1 ml 4% paraformaldehyde was added to each well of the 24-well plate that contained cells for 12 minutes at room temperature. Afterwards, collagen gels were washed twice with PBS and stored at 4° C. until staining. To permeabilize the cells, 0.5% triton-x (diluted in DPBS) was added to each well for 1 hour. The gels were then blocked with 0.1% triton-x, 10% fetal bovine serum (FBS), and 1% bovine serum albumin (BSA) for 3 hours at room temperature or overnight at 4° C. Primary antibodies were then diluted as instructed in the same blocking solution and added to the gels for 3 hours at room temperature or overnight at 4° C. The gels were then washed with DPBS 3 times for 10 minutes each. Secondary antibodies were diluted in 5% host serum (in DPBS) and added to the collagen gels for 1 hour. The gels were then washed 3 times with DPBS again. Confocal images were acquired using an Andor™ CSU-W confocal spinning disk on a Leica® DMi8 inverted microscope.

### Single Cell RNA Sequencing

Organoids were produced as described above and cultured for 48 hours in 2% Matrigel® organoid media. For the Oh timepoint, a subset of organoids were collected and dissociated to single cells by submersing in Accumax™ (StemCell Technologies™ #0792) for 10-15 minutes with occasional mixing by pipette. For the other two timepoints, organoids were plated in 3.75 mg/ml collagen I using the protocol above. At the appropriate time, organoids were removed from collagen gels using collagenase (1 mg/ml, MilliporeSigma® C2139) in DMEM/F12 for 5-10 minutes. Afterwards, organoids were dissociated to single cells in Accumax™ Dissociated cells were spun down, resuspended in PBS, and filtered through a 40 μm size filter. Cells were then counted using a countess, and resuspended in freezing media (Organoid media with 20% FBS and 10% DMSO) at a concentration of 1 million cells per 500 μL. Cells were shipped to Genewiz® (Azenta Life Sciences®) and sequenced on the 10× 3′ platform. Alignment and pre-processing were performed by Genewiz® using 10× protocols.

### Single Cell RNA Sequencing Data Analysis

Seurat V4 (Hao and Hao et al., 2021) was used for processing and analysis of count matrices generated by 10×. Cells containing fewer than 500 detected genes or more than 10% mitochondrial genes were filtered out to discard low quality and dead cells respectively. Data was then log-normalized using Seurat normalization with a scale factor of 2000. 2000 variable features were determined using Seurat vst method. Stromal cells were defined and filtered out using the markers Col1a1, Fabp4, and Epcam and removed from data to allow for analysis of epithelial tumor cells. After each replicate and timepoint were processed as stated above, timepoints from individual replicates were merged. For each timepoint-merged biological replicate, cell cycle regression was performed using Seurat, after which data was scaled using standard Seurat protocol. UMAP figures were made by performing Seurat principal component analysis with 20 principal components, followed by UMAP projection with uwot method. To find genes correlating with Krt14, biological replicates were merged, and then correlation was performed on a matrix of gene expression in each cell. The highest and lowest 1% of genes correlating with Krt14 were used as input for gene set enrichment analysis with Metascape. Gene module scores were made with gene set lists reported in Metascape after analyzing genes highly correlated with Krt14. Mouse cell type annotation was performed using scMCA.

### Public Dataset Analysis

Each additional dataset used for FIGS. 1A-1L, 6A-6I, and 7A-7K was processed in the same manner as the original data presented herein unless pre-processing had already occurred. For heatmap production, each dataset sample was summarized by average Krt14 expression and the correlation of Krt14 with each gene set module score. Values were plotted using ComplexHeatmap.

qPCR

Organoids were removed from collagen gels using collagenase (1 mg/ml, MilliporeSigma® C2139) in DMEM/F12 at 37° C. for 5-10 minutes. The organoids were then washed with PBS, and spun down at 300×g for 5 minutes. RNA was then extracted from the organoid pellet using the RNeasy™ kit (Qiagen®) according to manufacturer instructions. All RNA was quantified using a NanoDrop™ and stored at −80° C. cDNA was produced using the SuperScript™ IV VILO™ kit according to instructions. All qPCR primers were ordered from MilliporeSigma® (KiCqStart™ predesigned primers). qPCR was run with PowerUp™ SYBR™ Green master mix following instructions using a primer concentration of 200 nM.

### RNA-Seq

Organoids were embedded in 3D collagen and treated with either BMS493 at a concentration of 5 μM or AtRA at a concentration of 1 μM for 72 hours. Doses were chosen to capture maximal phenotypic changes while cell viability was maintained. RNA was extracted from organoids as described above for qPCR. RNA was sent to Genewiz® (Azenta Life Sciences®) for sequencing and processed as follows:

Total RNA samples were quantified using Qubit® 2.0 Fluorometer (Life Technologies®, Carlsbad, CA, USA) and RNA integrity was checked with 4200 TapeStation System™ (Agilent Technologies®, Palo Alto, CA, USA). Samples were initially treated with TURBO™ DNase (Thermo Fisher Scientific®, Waltham, MA, USA) to remove DNA contaminants. The next steps included performing rRNA depletion using QIAseq® FastSelect™-IRNA HMR kit (Qiagen®, Germantown, MD, USA), which was conducted following the manufacturer's protocol.

RNA sequencing libraries were constructed with the NEBNext® Ultra™ II RNA Library Preparation Kit for Illumina® by following the manufacturer's recommendations. Briefly, enriched RNAs are fragmented for 15 minutes at 94° C. First strand and second strand cDNA are subsequently synthesized. cDNA fragments are end repaired and adenylated at 3′ends, and universal adapters are ligated to cDNA fragments, followed by index addition and library enrichment with limited cycle PCR. Sequencing libraries were validated using the Tapestation 4200 System™ (Agilent Technologies®, Palo Alto, CA, USA), and quantified using Qubit® 2.0 Fluorometer (Thermo Fisher Scientific®, Waltham, MA, USA) as well as by quantitative PCR (KAPA Biosystems™, Wilmington, MA, USA). The sequencing libraries were multiplexed and clustered on one lane of a flowcell. After clustering, the flowcell was loaded on the Illumina® HiSeq™ 4000 instrument according to the manufacturer's instructions. The samples were sequenced using a 2×150 Pair-End (PE) configuration.

Image analysis and base calling were conducted by the HiSeq Control Software (HCS). Raw sequence data (.bcl files) generated from Illumina® HiSeq™ was converted into FASTQ files and de-multiplexed using Illumina's bcl2fastq 2.17 software. One mismatch was allowed for index sequence identification.

### RNA-Seq Analysis

Prior to alignment, RNA-seq reads were trimmed using Trim Galore to remove adapter sequences and low-quality bases, and read quality was confirmed using fastQC. Trimmed reads were aligned to the Mus musculus genome using STAR. Paired reads with both mates aligning to the genome were counted at the gene level using feature Counts for downstream differential expression analysis.

Differential expression analysis was performed following the limma-voom procedure. Prior to differential expression analysis, very low abundance genes were filtered using the filterByExpr function in edgeR with default parameters. Effective library sizes were calculated using the trimmed mean of M-values (TMM) normalization method and reads were processed in voom to generate log 2-transformed values and precision weights for limma. Using the limma, normalized expression values were fit to a random effects linear model of the form Expr~Treatment+Mouse, where Treatment denotes the retinoic acid treatment received, and Mouse denotes the identity of the mouse from which the organoids were derived. The differential expression between treatment groups were then obtained as contrasts using the contrasts.fit function in limma. Empirical Bayes smoothing was applied on both the linear model and fitted contrast coefficients using edgeR. False-discovery rates for differential expression at the gene-level were calculated with the Benjamini-Hochberg correction procedure using the toptable function in limma. Normalized transcript abundances are reported as the TMM-normalized counts-per-million obtained from the voom function in limma.

### In-Vivo Drug Studies

BMS493 treatment was based upon the work described in Viragova et al., 2023. A stock concentration of 50 mg/mL BMS493 (MedChemExpress®, HY-108529) was prepared in DMSO and kept at −20° C. in aliquots. On the day of treatment, the BMS493 was diluted to a final concentration of 2 mg/mL in 0.15M hydroxypropyl-b-cyclodextrin (Cayman Chemicals®, 16169). This was then given to mice via intraperitoneal injection (I.P.) at a final dose of 40 mg/kg (1 mg/25 g, adjusted for weight of each mouse). AtRA treatment followed the same general protocol. Stock concentration of AtRA was 15 mg/mL in DMSO which was diluted to 0.6 mg/ml in 0.15M hydroxypropyl-b-cyclodextrin. This was then given to mice via intraperitoneal injection (I.P.) at a final dose of 12 mg/kg. Vehicle control was made by dissolving an equal volume of DMSO in 0.15M hydroxypropyl-β-cyclodextrin. Mice were treated with BMS493, AtRA, or control 3 times a week (Monday, Wednesday, Friday) and monitored for negative effects. Side effects consistent with vitamin A deficiency were observed starting after one week of treatment, including eye swelling and secretions.

### In-Vivo Orthotopic Tumor Growth Experiments

Tumor organoids were collected from MTMG MMTV-PyMT mice as described above. The day before transplant, tumor organoids were dissociated to single cells by suspending in Accumax™ for 10 minutes. Cells were counted and allowed to re-aggregate into clusters for 24 hours in organoid media with 2% Matrigel®. The next day, spheroids were resuspended in a 1:1 Matrigel: DMEM/F12 at a concentration of 3 million cells/100 μL. 20 μL of this mixture (containing 600,000 cells) was surgically transplanted into the right T4 mammary fat pad of 8 week old female NSG mice. After 3 weeks, tumors were palpable and treatment began as described above. Tumors were measured 2×/week and estimated tumor volume was calculated with the formula V=(L*W2)/2.

### In-Vivo De-Novo Tumor Growth Experiments

Female MMTV-PyMT mice were monitored for palpable tumors, which typically appear at ages 8-10 weeks. At the beginning of the experiment, age-matched mice were split into two treatment groups. Mice were treated as described above three times a week. On each day of treatment, up to 4 tumors were measured per mouse (upper and lower, left and right). At the end of treatment the mice were euthanized. One tumor was flash frozen in liquid nitrogen for RNA collection. The remaining tumors were collected and fixed in either 4% paraformaldehyde (PFA) or 10% neutral buffered formalin (NBF) for 72 hours. PFA fixed tumors were embedded in OCT and kept at −80° C. until sectioning. NBF fixed tumors were processed, paraffin embedded, and sectioned by the Experimental Histopathology core at the Fred Hutchinson Cancer Center.

### In-Vivo Tail-Vein Metastasis Experiments

For tail-vein metastasis experiments, mTomato-MMTV-PyMT organoids were dissociated to single cells at day 0 using Accumax™ (20 minutes at 37° C.). To generate clusters, single cells were plated in non-adherent dishes at 150,000 cells/mL in media+2% basement membrane-rich gel (v/v). After 24 hours, the clusters were resuspended in PBS at a concentration of 1 million cells/mL using cell counts from the day before. 200,000 cells in 200 uL of PBS were injected into Nod scid gamma (NSG) immunocompromised mice. For the de-novo study, mice were treated following the treatment protocol described above for 2 weeks, after which mice were euthanized and lungs imaged under a dissecting microscope for quantification of fluorescent (metastatic) area. Lungs were then fixed in 4% paraformaldehyde for 48 hours, then then transferred to 25% sucrose in DPBS overnight at 4° C. before embedding in OCT and storing at −80° C.

### 2D Histology

H&E and immunohistochemistry (IHC) stains were performed by the experimental histology core at Fred Hutchinson Cancer Center. Slides were scanned on a Ventana DP® 200 slide scanner. Images were analyzed in qupath.

TABLE 1
Primers, antibodies, and plasmids used in Example 1

| | | |
|---|---|---|
| Resource | Source | Identifier |

Antibodies

| | | |
|---|---|---|
| Keratin 14 | Biolegend ® | 906004 |
| Keratin 17 | Cell Signaling ® | 4543S |
| Hair Keratin (AE13) | Santa Cruz ® | sc-57012 |
| Loricrin | Abcam ® | ab85679 |
| pHistone H3 | Millipore ® | 06-570 |
| Cleaved caspase 3 | Cell Signaling ® | 9579 |
| Phalloidin 568 | Thermo Fisher ® | A12380 |

Recombinant DNA

| | | |
|---|---|---|
| MD2.G | Gift from Didier Trono | Addgene #12259 |
| PsPax2 | Gift from Didier Trono | Addgene #12260 |
| EF1a Tet On (ETO) | Gift from Emily Hatch | Addgene #84776 |

### Code and Data Availability

To recreate analyses, see github for R, cellprofiler, and qupath scripts. The following data will be deposited in the NCBI Gene Expression Ombnibus (GEO): Fastq and processed Seurat object for scRNA-seq of invading MMTV-PyMT organoids, Fastq and processed counts for RNAseq of MMTV-PyMT organoids.

Additional scRNA-seq datasets used are available at the Gene Expression Omnibus accession numbers listed below:
FVB mammary gland: GSE103275
C3 (1) tag organoids: GSE149299
MMTV-PyMT tumor: GSE214815
C3 (1 818) tag tumor: GSE182389
Pten-Myc tumor: GSE215070
Human BRCA dataset #1: GSE161529
Human BRCA dataset #2: GSE176078

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of inducing irreversible epidermal differentiation in a basal-like tumor cell, the method comprising:
contacting the basal-like tumor cell with an effective amount of at least one agent that modulates retinoic acid signaling,
wherein the basal-like tumor cell expresses one or more epidermal genes, and wherein the method induces directed differentiation of the basal-like tumor cell towards a terminal epidermal phenotype.

2. The method of claim 1, wherein the basal-like tumor cell is a carcinoma cell.

3. The method of claim 1, wherein the one or more epidermal genes comprise genes expressed in basal epidermal keratinocytes, and wherein the one or more epidermal genes are associated with cornified envelope formation, epidermal differentiation, and epidermal morphogenesis.

4. The method of claim 1, wherein the one or more epidermal genes comprise cytokeratin-14 (K14), K17, Stratifin (SFN), PERP, DSG3, and LAMA3, Krt6a, and Tp63.

5. The method of claim 1, wherein the basal-like tumor cell is derived from a squamous cell carcinoma, breast carcinoma, prostate carcinoma, or pancreatic carcinoma.

6. The method of claim 1, wherein the basal-like tumor cell is in vitro, ex vivo, or in vivo.

7. The method of claim 1, wherein the basal-like tumor cell is a breast carcinoma cell, and wherein the breast carcinoma cell is a triple negative breast carcinoma cell lacking expression of estrogen receptor (ER), progesterone receptor (PR), and HER2/Neu.

8. The method of claim 1, wherein the at least one agent modulates retinoic acid signaling by antagonizing a retinoic acid receptor, a retinoic acid signaling component, or a combination thereof.

9. The method of claim 1, wherein the at least one agent is selected from a small molecule, biologic, nucleic acid, and an antibody or a derivative thereof.

10. The method of claim 9, wherein the nucleic acid is an siRNA, shRNA, antisense-oligonucleotide, or a CRISPR-based gene editing system.

11. The method of claim 8, wherein the at least one agent modulates retinoic acid signaling by antagonizing a retinoic acid receptor, and wherein the retinoic acid receptor is RAR-α, RAR-β, and RAR-γ.

12. The method of claim 11, wherein the at least one agent is a small molecule, and wherein the small molecule is selected from BMS493, BMS614, LE135, and LY2955303.

13. The method of claim 8, wherein the at least one agent modulates retinoic acid signaling by antagonizing a retinoic acid receptor, and wherein the retinoic acid receptor is the RAR-α, and wherein the agent is BMS614.

14. The method of claim 1, wherein the terminal epidermal-like phenotype is characterized by one or more of decreased proliferation, reduced migration, or loss of invasive potential of the basal-like tumor cell relative to a basal-like tumor cell not contacted with the agent.

15. A method of treating a tumor in a subject, comprising:
administering to the subject a therapeutically effective amount of a composition comprising at least one agent capable of inducing an irreversible differentiation of the tumor towards a terminal epidermal phenotype,
wherein the tumor comprises basal-like tumor cells expressing one or more epidermal genes, wherein the one or more epidermal genes comprise genes associated with cornified envelope formation, epidermal differentiation, and epidermal morphogenesis.

16. The method of claim 15, wherein the one or more epidermal genes comprise cytokeratin-14 (K14), K17, Stratifin (SFN), PERP, DSG3, LAMA3, Krt6a, and Tp63.

17. The method of claim 15, wherein the at least one agent capable of inducing an irreversible differentiation of the tumor is an agent that modulates retinoic acid signaling.

18. The method of claim 15, wherein the terminal epidermal phenotype is characterized by an increased expression of one or more epidermal differentiation markers, and optionally a reduced expression of one or more basal-like tumor cell markers relative to a basal-like tumor cell of an untreated subject.

19. The method of claim 15, wherein the terminal epidermal phenotype is further characterized by one or more of upregulation of desmosomal adhesion, keratinization, and adhesion plaques relative to a basal-like tumor cell of an untreated subject.

20. The method of claim 17, wherein the at least one agent modulates retinoic acid signaling by antagonizing a retinoic acid receptor, a retinoic acid signaling component, or a combination thereof.

21. The method of claim 20, wherein the at least one agent is selected from a small molecule, biologic, nucleic acid, and an antibody or a derivative thereof.

22. The method of claim 21, wherein the nucleic acid is a siRNA, shRNA, antisense-oligonucleotide, or a CRISPR-based gene editing system.

23. The method of claim 17, wherein the at least one agent modulates retinoic acid signaling by antagonizing a retinoic acid receptor, and wherein the retinoic acid receptor is RAR-α, RAR-β, and RAR-γ.

24. The method of claim 23, wherein the at least one agent is a small molecule, and wherein the small molecule is selected from BMS493, BMS614, LE135, and LY2955303.

25. The method of claim 17, wherein the at least one agent modulates retinoic acid signaling by antagonizing a retinoic acid receptor, and wherein the retinoic acid receptor is the RAR-α, and wherein the agent is BMS614.

26. The method of claim 15, wherein the method is effective in reducing tumor growth, metastases, and inducing tumor regression.

27. The method of claim 15, further comprising administering a chemotherapeutic agent, immunotherapeutic agent, or radiation therapy.

28. The method of claim 27, wherein the method is effective in sensitizing the tumor to the chemotherapeutic agent.

29. The method of claim 27, wherein the method reduces metastatic spread of the tumor to a secondary tissue.

30. The method of claim 15, wherein the tumor is a squamous cell carcinoma, basal-like breast carcinoma, prostate carcinoma, or pancreatic carcinoma.

31. The method of claim 30, wherein the tumor is a breast tumor, and wherein the breast tumor is a triple negative breast tumor lacking expression of estrogen receptor (ER), progesterone receptor (PR), and Her-2/Neu.

32. The method of claim 15, wherein the subject is human.

33. A pharmaceutical composition comprising: at least one agent capable of inducing a directed and irreversible differentiation of basal-like tumor cells towards a terminal epidermal phenotype; and a pharmaceutically acceptable carrier.

34. The composition of claim 33 wherein the at least one agent capable of inducing a directed and irreversible differentiation of the basal-like tumor cells towards a terminal epidermal phenotype is an agent that modulates retinoic acid signaling.

35. A method of selectively treating a triple negative breast tumor in a subject, the method comprising:
obtaining a biological sample of the breast tumor from the subject;
detecting basal-like tumor cells expressing a unique transcriptional signature in the biological sample; and
administering to the subject a therapeutically effective amount of a composition comprising at least one agent capable of inducing irreversible differentiation of the basal-like tumor cells towards a terminal epidermal phenotype,
wherein the unique transcriptional signature comprises one or more epidermal genes expressed in basal epidermal keratinocytes associated with cornified envelope formation, epidermal differentiation, and epidermal morphogenesis.

36. The method of claim 35, wherein the one or more epidermal genes comprise cytokeratin-14 (K14), K17, Stratifin (SFN), PERP, DSG3, LAMA3, Krt6a, and Tp63.

37. The method of claim 35, wherein the at least one agent capable of inducing a directed and irreversible differentiation of the basal-like tumor cells is an agent that modulates retinoic acid signaling.

38. The method of claim 37, wherein the at least one agent modulates retinoic acid signaling by antagonizing a retinoic acid receptor, a retinoic acid signaling component, or a combination thereof.

39. The method of claim 37, wherein the at least one agent is a small molecule, biologic, nucleic acid, and an antibody, or a derivative thereof.

40. The method of claim 39, wherein the at least one agent is a nucleic acid, wherein the nucleic acid is an siRNA, shRNA, antisense-oligonucleotide, or a CRISPR-based gene editing system.

41. The method of claim 37, wherein the at least one agent modulates retinoic acid signaling by antagonizing a retinoic acid receptor, and wherein the retinoic acid receptor is RAR-α, RAR-β, and RAR-γ.

42. The method of claim 41, wherein the at least one agent is a small molecule, and wherein the small molecule is selected from BMS493, BMS614, LE135, and LY2955303.

43. The method of claim 37, wherein the at least one agent modulates retinoic acid signaling by antagonizing a retinoic acid receptor, and wherein the retinoic acid receptor is the RAR-α, and wherein the agent is BMS614.

44. The method of claim 35, further comprising administering to the subject at least one other therapeutic agent, wherein the at least one other therapeutic agent is selected from a chemotherapeutic agent, an immunotherapeutic agent, and radiation therapy.

45. The method of claim 35, wherein the terminal epidermal phenotype is characterized by an increased expression of one or more epidermal differentiation markers, optionally a reduced expression of one or more basal-like tumor cell markers relative to an untreated subject.

46. The method of claim 45, wherein the terminal epidermal phenotype is further characterized by upregulation of desmosomal adhesion, keratinization, and adhesion plaques relative to an untreated subject.

47. The method of claim 34, wherein the method is effective in reducing tumor growth, metastases, and inducing tumor regression.

48. A method of predicting susceptibility of a tumor in a subject to an agent capable of inducing differentiation of the tumor towards a terminal epidermal phenotype, the method comprising:
obtaining a biological sample of the tumor from the subject; and
detecting presence of basal-like tumor cells harboring a unique transcriptional signature in the biological sample,
wherein detection of the basal-like tumor cells harboring the unique transcriptional signature in the biological sample predicts that the tumor is susceptible to the agent.

49. The method of claim 47, wherein the unique transcriptional signature comprises one or more epidermal genes associated with cornified envelope formation, epidermal differentiation, and epidermal morphogenesis.

50. The method of claim 48, wherein the one or more epidermal genes comprise cytokeratin-14 (K14), K17, Stratifin (SFN), PERP, DSG3, LAMA3, Krt6a, and Tp63.

51. The method of claim 48, wherein the agent induces a directed and irreversible differentiation of the basal-like tumor cells towards the terminal epidermal phenotype.

52. The method of claim 51, wherein the agent modulates retinoic acid signaling.

53. The method of claim 52, wherein the agent modulates retinoic acid signaling by antagonizing a retinoic acid receptor, a retinoic acid signaling component, or a combination thereof.

54. The method of claim 48, wherein the tumor is a squamous cell carcinoma, basal-like breast carcinoma, prostate carcinoma, or pancreatic carcinoma.
